(19) United States
(12) Patent Application Publication
(10) Pub. No.: US 2024/0131147 A1
(43) Pub. Date: Apr. 25, 2024

(54) POLYPEPTIDES MIMICKING MPER AND V3-LOOP HIV-1 ENV GLYCOPROTEIN EPITOPES

(71) Applicants: UNIVERZITA PALACKEHO V OLOMOUCI, Olomouc (CZ); NEXIUM, S.R.O., Chlumec nad Cidlinou (CZ)

(72) Inventors: Petr MALY, Praha 4 (CZ); Milan RASKA, Olomouc (CZ); Milan KUCHAR, Sobulky (CZ); Petr KOSZTYU, Olomouc (CZ); Jiri CERNY, Plzen (CZ); Veronika DANIEL LISKOVA, Praha 11 (CZ); Hana PETROKOVA, Praha 10 (CZ)

(21) Appl. No.: 18/278,208

(22) PCT Filed: Feb. 18, 2022
(86) PCT No.: PCT/CZ2022/050017

§ 371 Date: Aug. 21, 2023

(30) Foreign Application Priority Data
Feb. 26, 2021 (EP) 21159446.0

(51) Int. Cl. A61K 39/21; A61P 31/18; C07K 14/005

(52) U.S. Cl. CPC A61K 39/21; A61P 31/18; C07K 14/005; A61K 2039/575

(57) ABSTRACT

The present invention provides polypeptides having a length of up to 180 amino acids and containing a sequence selected from sequences identical or differing at most in 5 amino acids from the sequence: KSELAVEILEKGQVRFWMQAX₂₁X₂₂X₂₃X₂₄GNAKVNYIFNEKEIFEGPKYKMHIDXsoXsiXszGIIEM FMEKLQDEDEGTYTFQLQX₇₆X₇₇X₇₈X₇₉X₈₀NHSTVVLVGDVFKKLQKEAEFQRQEWIRKQG (SEQ ID NO. 2), wherein X21X22X23X24 is selected from KAQQ (SEQ ID NO. 33), LSVF (SEQ ID NO. 34), ATPS (SEQ ID NO. 35), EIMW (SEQ ID NO. 36), DGSS (SEQ ID NO. 37), LLPL (SEQ ID NO. 38), WMWW (SEQ ID NO. 39), MNLY (SEQ ID NO. 40), MWRN (SEQ ID NO. 41), IMME (SEQ ID NO. 42), KHQL (SEQ ID NO. 43), HWQF (SEQ ID NO. 44), YAGN (SEQ ID NO. 45) and HGQW (SEQ ID NO. 46); X50X51X52 is selected from RNT, IMF, GHE, PSW, RAN, YFW, ITL, QAM, DMR, WLW, QGE, VQY and VSL; X76X77X78X79X80 is selected from SHHLG (SEQ ID NO. 47), FMLMM (SEQ ID NO. 48), VILIL (SEQ ID NO. 49), IVTPL (SEQ ID NO. 50), DFIIW (SEQ ID NO. 51), MWSE (deletion) (SEQ ID NO. 52), LYYAW (SEQ ID NO. 53), MMIEY (SEQ ID NO. 54), WMTQT (SEQ ID NO. 55), PQLWL (SEQ ID NO. 56), EPIFL (SEQ ID NO. 57) and QTATY (SEQ ID NO. 58); and optionally further having an affinity tag attached to the N-terminus or to the C-terminus of the sequence. These polypeptides mimic HIV-1 Env glycoprotein epitopes and are useful in preparation of antibodies and vaccines.

### FIELD OF THE INVENTION

The present invention relates to polypeptides derived from the structure of human contractile protein myomesin-1 domain. The polypeptides of the present invention include three groups of polypeptides designated MLA, MLB and MLD targeted to 10E8, PGT126, and PGT121 broadly neutralizing antibodies (bNAbs) against HIV-1 virus, respectively. The polypeptides recognize a paratope of the particular HIV-1 bNAb and mimic the cognate epitopes of Env glycoprotein and are, therefore, suitable as immunogens for stimulation of production of HIV-1-neutralizing antibodies and for the development of a vaccine preventing HIV infection.

### BACKGROUND ART

Stimulation of protective immunity by the production of broadly neutralizing antibodies (bNAbs) in immunized individuals remains a challenge in HIV-1 vaccine development. Identification and characterization of highly potent bNAbs cloned from B-cells of elite neutralizers provide a molecular clue for designing new vaccine strategies. The main target of neutralizing antibody on the HIV-1 virus surface is the envelope glycoprotein (Env), heterotrimer composed of three identical gp120/gp41 subunits. Gp120 protrudes from virus surface whereas gp41 is transmembrane subunit. Systematic studies of specificity, binding affinity, breadth and potency led to a clustering of bNAbs into four major groups specific for V2 loop of HIV-1 Env glycoproteins (V2 bNAbs), V3 glycan (V3 bNAbs), CD4 binding site (CD4 bNAbs) and membrane-proximal external region (MPER bNAbs) (Moore, P. L. The Neutralizing Antibody Response to the HIV-1 Env Protein. Curr HIV Res 16, 21-28, 2018). Some of these bNAbs exhibit extraordinary breadth and potency and efficiently neutralize viral infection of host cells and represent exclusive candidates for vaccine design and therapy (Sok, D., and Burton, D. R. Recent progress in broadly neutralizing antibodies to HIV. Nat Immunol 19, 1179-1188, 2018).

To overcome persisting problems with low-efficient immunization by Env/gp120 and Env-modified glycan-carrying immunogens, we recently proposed a novel strategy that is based on immunization by artificial scaffold proteins mimicking HIV-1 Env epitope (Kosztyu, P., Kuchar, M., Cerny, J., Barkocziova, L., Maly, M., Petrokova, H., Czernekova, L., Liskova, V., Raskova Kafkova, L., Knotigova, P., et al., Proteins mimicking epitope of HIV-1 virus neutralizing antibody induce virus-neutralizing sera in mice. EBioMedicine 47, 247-256, 2019). These protein variants selected from a highly complex albumin-binding domain-derived combinatorial library by directed evolution can function as “protein imprints” of paratopes of the most-potent HIV-1 bNAbs. In our proof-of-concept study, we demonstrated that variants called VRA017, VRA019 and VRA177 mimicking epitope of VRC01 bNAb elicited virus-neutralizing sera in mice as tested on a panel of pseudotyped HIV-1 viruses on reporter TZM-bl cells. The VRA polypeptides mimic the epitope of a principle CD4 binding site required for the initial interaction of HIV-1 virus with host cell membrane and elicit antibodies targeting gp120 subunit. To strengthen the efficacy of the neutralization, however, it is still desirable to develop a more complex approach with production of polypeptides that would elicit antibodies blocking other crucial gp41 and gp120 epitopes within the HIV-1 envelope glycoprotein to avoid virus mutants escape from the immune surveillance.

### DISCLOSURE OF THE INVENTION

The present invention provides polypeptides called herein MLA suitable for induction of HIV-1 virus-neutralizing antibodies which target “super candidate” bNAb 10E8 that is specific for MPER of gp41 subunit protruding from the viral envelope. The present invention also provides polypeptides called herein MLB and MLD that mimic V3-loop epitopes of highly potent neutralizing antibodies PGT126 and PGT121, respectively. The all polypeptides were obtained from a loop-randomized scaffold library designed on the structure of domain 10 of human contractile muscle protein myomesin-1. When the novel polypeptides are used as immunogens in experimental mice to stimulate the production of specific antibodies, the Env-specificity and virus-neutralizing activity of the hyperimmune sera tested on a panel of pseudotyped HIV-1 viruses of clades A, B, C, D and AE result in neutralization of most of the tested pseudoviruses in vitro.

The sequence of the parental human myomesin-1 domain 10 protein (PDB ID 6T3O) is as follows:

(SEQ ID NO. 1)
KSELAVEILEKGQVRFWMQAEKLSGNAKVNYIFNEKEIFE
GPKYKMHIDRNTGIIEMFMEKLQDEDEGTYTFQLQDGKAT
NHSTVVLVGDVFKKLQKEAEFQRQEWIRKQG

Amino acid residues in positions 21-24, 50-52 and 76-80 were randomized. These positions are shown in bold in SEQ IN NO. 1.

The present invention thus provides polypeptides having the length of up to 180 amino acids and containing a sequence selected from sequences identical or differing at most in 5 amino acids from the sequence:

(SEQ ID NO. 2)
KSELAVEILEKGQVRFWMQAX₂₁X₂₂X₂₃X₂₄GNAKVNYIFNEKEIFE
GPKYKMHIDX₅₀X₅₁X₅₂GIIEMFMEKLQDEDEGTYTFQLQX₇₆X₇₇X₇₈
X₇₉X₈₀NHSTVVLVGDVFKKLQKEAEFQRQEWIRKQG,

wherein
X₂₁X₂₂X₂₃X₂₄ is selected from KAQQ (SEQ ID NO. 33), LSVF (SEQ ID NO. 34), ATPS (SEQ ID NO. 35), EIMW (SEQ ID NO. 36), DGSS (SEQ ID NO. 37), LLPL (SEQ ID NO. 38), WMWW (SEQ ID NO. 39), MNLY (SEQ ID NO. 40), MWRN (SEQ ID NO. 41), IMME (SEQ ID NO. 42), KHQL (SEQ ID NO. 43), HWQF (SEQ ID NO. 44), YAGN (SEQ ID NO. 45) and HGQW (SEQ ID NO. 46); X₅₀X₅₁X₅₂ is selected from RNT, IMF, GHE, PSW, RAN, YFW, ITL, QAM, DMR, WLW, QGE, VQY and VSL;
X₇₆X₇₇X₇₈X₇₉X₈₀ is selected from SHHLG (SEQ ID NO. 47), FMLMM (SEQ ID NO. 48), VILIL (SEQ ID NO. 49), IVTPL (SEQ ID NO. 50), DFIIW (SEQ ID NO. 51), MWSE (deletion) (SEQ ID NO. 52), LYYAW (SEQ ID NO. 53), MMIEY (SEQ ID NO. 54), WMTQT (SEQ ID NO. 55), PQLWL (SEQ ID NO. 56), EPIFL (SEQ ID NO. 57) and QTATY (SEQ ID NO. 58), optionally further having an affinity tag attached to the N-terminus or to the C-terminus of the sequence.

In some embodiments of the invention, the following combinations of the variables in SEQ ID NO. 2 are preferred:
X₂₁X₂₂X₂₃X₂₄ is selected from KAQQ (SEQ ID NO. 33), LSVF (SEQ ID NO. 34), ATPS (SEQ ID NO. 35), EIMW (SEQ ID NO. 36), DGSS (SEQ ID NO. 37), LLPL (SEQ ID NO. 38), WMWW (SEQ ID NO. 39), and MNLY (SEQ ID NO. 40);
X₅₀X₅₁X₅₂ is selected from RNT, IMF, GHE, PSW, RAN, YFW, ITL and QAM;
X₇₆X₇₇X₇₈X₇₉X₈₀ is selected from SHHLG (SEQ ID NO. 47), FMLMM (SEQ ID NO. 48), VILIL (SEQ ID NO. 49), IVTPL (SEQ ID NO. 50), DFIIW (SEQ ID NO. 51), MWSE (deletion) (SEQ ID NO. 52), LYYAW (SEQ ID NO. 53) and MMIEY (SEQ ID NO. 54).

Hereinafter, polypeptides having these variables are called MLA polypeptides.

In some embodiments of the invention, the following combinations of the variables in SEQ ID NO. 2 are preferred:
X₂₁X₂₂X₂₃X₂₄ is selected from MWRN (SEQ ID NO. 41), IMME (SEQ ID NO. 42) and KHQL (SEQ ID NO. 43);
X₅₀X₅₁X₅₂ is selected from RNT, DMR and WLW;
X₇₆XX₇₈X₇₉X₈₀ is selected from IVTPL (SEQ ID NO. 50) and WMTQT (SEQ ID NO. 55).

Hereinafter, polypeptides having these variables are called MLB polypeptides.

In some embodiments of the invention, the following combinations of the variables in SEQ ID NO. 2 are preferred:
X₂₁X₂₂X₂₃X₂₄ is selected from HWQF (SEQ ID NO. 44), YAGN (SEQ ID NO. 45) and HGQW (SEQ ID NO. 46);
X₅₀X₅₁X₅₂ is selected from QGE, VQY and VSL;
X₇₆XX₇₈X₇₉X₈₀ is selected from PQLWL (SEQ ID NO. 56), EPIFL (SEQ ID NO. 57) and QTATY (SEQ ID NO. 58).

Hereinafter, polypeptides having these variables are called MLD polypeptides.

Particularly preferred polypeptides of the present invention have the length of up to 180 amino acids and contain sequence selected from sequences identical or differing at most in 5 amino acids from the sequences:

(SEQ ID NO. 3)
KSELAVEILEKGQVRFWMQAKAQQGNAKVNYIFNEKEIFE
GPKYKMHIDIMFGIIEMFMEKLQDEDEGTYTFQLQSHHLG
NHSTVVLVGDVFKKLQKEAEFQRQEWIRKQG
(SEQ ID NO. 4)
KSELAVEILEKGQVRFWMQALSVFGNAKVNYIFNEKEIFE
GPKYKMHIDRNTGIIEMFMEKLQDEDEGTYTFQLQFMLMM
NHSTVVLVGDVFKKLQKEAEFQRQEWIRKQG
(SEQ ID NO. 5)
KSELAVEILEKGQVRFWMQAATPSGNAKVNYIFNEKEIFE
GPKYKMHIDGHEGIIEMFMEKLQDEDEGTYTFQLQVILIL
NHSTVVLVGDVFKKLQKEAEFQRQEWIRKQG
(SEQ ID NO. 6)
KSELAVEILEKGQVRFWMQAEIMWGNAKVNYIFNEKEIFE
GPKYKMHIDPSWGIIEMFMEKLQDEDEGTYTFQLQIVTPL
NHSTVVLVGDVFKKLQKEAEFQRQEWIRKQG
(SEQ ID NO. 7)
KSELAVEILEKGQVRFWMQADGSSGNAKVNYIFNEKEIFE
GPKYKMHIDRANGIIEMFMEKLQDEDEGTYTFQLQDFIIW
NHSTVVLVGDVFKKLQKEAEFQRQEWIRKQG
(SEQ ID NO. 8)
KSELAVEILEKGQVRFWMQALLPLGNAKVNYIFNEKEIFE
GPKYKMHIDYFWGIIEMFMEKLQDEDEGTYTFQLQMWSEN
HSTVVLVGDVFKKLQKEAEFQRQEWIRKQG
(SEQ ID NO. 9)
KSELAVEILEKGQVRFWMQAWMWWGNAKVNYIFNEKEIFE
GPKYKMHIDITLGIIEMFMEKLQDEDEGTYTFQLQLYYAW
NHSTVVLVGDVFKKLQKEAEFQRQEWIRKQG
(SEQ ID NO. 10)
KSELAVEILEKGQVRFWMQAMNLYGNAKVNYIFNEKEIFE
GPKYKMHIDQAMGIIEMFMEKLQDEDEGTYTFQLQMMIEY
NHSTVVLVGDVFKKLQKEAEFQRQEWIRKQG
(SEQ ID NO. 11)
KSELAVEILEKGQVRFWMQAMWRNGNAKVNYIFNEKEIFE
GPKYKMHIDRNTGIIEMFMEKLQDEDEGTYTFQLQWMTQT
NHSTVVLVGDVFKKLQKEAEFQRQEWIRKQG
(SEQ ID NO. 12)
KSELAVEILEKGQVRFWMQAIMMEGNAKVNYIFNEKEIFE
GPKYKMHIDDMRGIIEMFMEKLQDEDEGTYTFQLQIVTPL
NHSTVVLVGDVFKKLQKEAEFQRQEWIRKQG
(SEQ ID NO. 13)
KSELAVEILEKGQVRFWMQAKHQLGNAKVNYIFNEKEIFE
GPKYKMHIDWLWGIIEMFMEKLQDEDEGTYTFQLQIVTPL
NHSTVVLVGDVFKKLQKEAEFQRQEWIRKQG
(SEQ ID NO. 14)
KSELAVEILEKGQVRFWMQAHWQFGNAKVNYIFNEKEIFE
GPKYKMHIDQGEGIIEMFMEKLQDEDEGTYTFQLQPQLWL
NHSTVVLVGDVFKKLQKEAEFQRQEWIRKQG
(SEQ ID NO. 15)
KSELAVEILEKGQVRFWMQAYAGNGNAKVNYIFNEKEIFE
GPKYKMHIDVQYGIIEMFMEKLQDEDEGTYTFQLQEPIFL
NHSTVVLVGDVFKKLQKEAEFQRQEWIRKQG
and
(SEQ ID NO. 16)
KSELAVEILEKGQVRFWMQAHGQWGNAKVNYIFNEKEIFE
GPKYKMHIDVSLGIIEMFMEKLQDEDEGTYTFQLQQTATY
NHSTVVLVGDVFKKLQKEAEFQRQEWIRKQG,

optionally further having an affinity tag attached to the N-terminus or to the C-terminus of the sequence.

In some embodiments, the sequences of the present invention are identical to sequence SEQ ID NO. 2 (with the listed variable regions), or to sequences SEQ ID NO. 3 to SEQ ID NO. 16, optionally further having an affinity tag attached to the N-terminus or to the C-terminus of the sequence.

In some embodiments, the sequences of the present invention differ in at most 5 amino acids from sequence SEQ ID NO. 2 (with the listed variable regions), or from sequences SEQ ID NO. 3 to SEQ ID NO. 16, and optionally further have an affinity tag attached to the N-terminus or to the C-terminus of the sequence.

The term “differing at most in 5 amino acids” or “differ in at most 5 amino acids” means that 1 or 2 or 3 or 4 or 5 amino acids in the sequence are replaced (substituted) by other amino acids.

The term “have a length of up to 180 amino acids” means that the sequence SEQ ID NO. 2 (with the listed variable regions), or sequences SEQ ID NO. 3 to SEQ ID NO. 16, may be extended on C-terminus or on N-terminus or on both termini with a maximum total of 69 amino acids (or 70 amino acids). Preferably, the length of the polypeptide may be up to 160 or up to 140 or up to 120 amino acids.

In the present invention it has been found that polypeptides of this invention preferentially bind to variable regions of HIV-1 broadly neutralizing monoclonal antibodies 10E8 (MLA polypeptides), PGT126 (MLB polypeptides) and PGT121 (MLD polypeptides) as demonstrated by a higher binding affinity to these bNAbs in comparison to IgG isotype control and by competition assays. Use of polypeptides can be affected by a combination of an initial immunization dose and further booster doses, in which different versions of the polypeptides due to a substantial shape surface similarity can significantly increase production of neutralizing antibodies raised against the particular epitope-mimicking immunogen.

Affinity tags are typically used for purification of produced polypeptides, and then they can be cleaved or maintained in the sequence. Polypeptide tags may include, for example: ALFA-tag, AviTag, C-tag, polyglutamate tag, polyarginine tag, E-tag, FLAG-tag, HA-tag, His-tag, Myc-tag, NE-tag, Rho1D4-tag, S-tag, Softag1, Softag3, Spot-tag, Strep-tag, T7-tag, TC tag, Ty tag, V5 tag, VSV-tag, Xpress tag, Isopeptag, SpyTag, SnoopTag, Dog-Tag, SdyTag. Preferred tags are poly(his), FLAG, AviTag, HA, Myc, S-tag or V5-tag.

Polypeptides in the present invention are appropriate for the use in pharmaceutical technology, especially as mimicking recombinant protein ligands exploitable for development of more efficient vaccine preventing HIV-1 virus infection. To this goal it is beneficial to attach other helper proteins to the mentioned polypeptides of the present invention that can stimulate antibody production, for example serum albumin or heat shock protein hsp70. These helper proteins can be covalently linked to polypeptides forming a chimeric protein. Further possibilities are to modify the mentioned polypeptides by an attachment of helper N- or C-terminal sequences (tags), which allow their specific detection or their oriented immobilization to surface of carries such as nanoliposomes with enhancement of immunization efficacy. Such tags include, in preferred embodiments, affinity or detection tags such as poly(his), FLAG, AviTag, HA, Myc, S-tag or V5-tag.

Polypeptides in the present invention defined by amino acid sequence stimulate production of serum antibodies after being used for immunization of experimental animals. Hyperimmune sera of immunized animals suppressed infection of reporter cells by tested Env-pseudotyped viruses in the model system and this represents one of the key mechanisms of HIV-1 infection control and one of the aims for development of a preventative vaccine that is still not available in the market.

The major advantage of these polypeptides, in comparison to vaccines currently being tested, is their mimicking complexity for three different HIV-1 Env epitopes located at MPER of gp 41 and V3-loop of gp120. This allows neutralizing distinct prominent epitopes defined by the most efficient HIV-1 bNAbs 10E8, PGT121 and PGT126. Simultaneous elicitation of a portfolio of neutralizing antibodies by a host prevents virus mutants' escape from the immune surveillance. Practical advantage of these polypetides is their easy preparation, stability and absence of posttranslational modifications and this allows their easy biotechnological production in prokaryotic host cells Escherichia coli and their further utilization as vaccine antigens.

The present invention thus also provides a vaccine for prevention of HIV-1 virus infection, comprising polypeptide according to the present invention and/or conjugate according to the present invention as an active ingredient or as an auxiliary ingredient. In some embodiments, the vaccine may contain at least two polypeptides according to the present invention and/or at least two conjugates according to the present invention. Suitable auxiliary substances for formulation of vaccines are known in the art.

The present invention further provides a DNA sequence selected from the group comprising complementary DNA coding for the amino acid sequence of the polypeptides of the present invention

The present invention further includes the use of said DNA sequence for the preparation of polypeptides or recombinant proteins produced in bacterial, yeast, insect, mammal or human host cells, and also these host cells, containing at least one DNA sequence of the present invention.

### BRIEF DESCRIPTION OF DRAWINGS

FIG. 1. Production characterization of human myomesin-1 domain 10. A) Analysis of protein fractions of human myomesin-1 domain 10 using SDS-PAGE gel electrophoresis, fractions from gel filtration chromatography on Superdex 75 10/300, M—molecular weight marker. B) Thermal stability of human myomesin-1 domain 10 measured by thermofluor thermal shift assay. Normalized thermal melting fluorescence curve (full line) and the first derivative of fluorescence curve versus temperature (dashed line). The melting point is given as the lowest point of the dashed curve.

FIG. 2. Human Myomesin-1 Domain 10, a Loop-type Myomedin library concept. A) The crystal structure of the myomesin-1 domain 10 used for randomization (PDB ID 6t3o) as a part of the structures of a longer myomesin fragment (PDB ID 2y23). The domain 10 is shown with the randomized residues indicated as black spheres. B) A rotated view on domain 10 is shown in cartoon representation with the randomized residues indicated as black spheres.

FIG. 3. Binding of MLA protein variants to 10E8 bNAb, IgG isotype control and BSA. Myomedin variants of eight selected MLA clones in the form of purified fusion proteins with N-terminal polyhistidine tag and C-terminal V5 tag produced in E. coli BL21 (DE3) were assayed in ELISA, the parental non-randomized Myomedin was used as a negative control. Binding to immobilized 10E8 bNAb (labeled as 10E8 IgG), IgGli isotype (labeled as IgG lambda) and BSA was detected by anti-V5 Ab-HRP conjugate. Each point is shown as the mean value of triplicates with standard deviation.

FIG. 4. Immunization of experimental mice with a panel of MLA variants induced serum antibodies specifically recognizing HIV-1 Env on the virus surface. A) Mice were immunized by the administration of four doses of individual MLA variants including wild-type (MyoWT). Sera were collected and tested in their reactivity with non-replicative HIV-1 viruses pseudotyped with B) Clade C Env (ZM109F) or C) Clade B Env (AC10.0, QH0692, RHPA) coated on ELISA plates. Statistical comparison was performed by ANOVA Kruskal-Wallis test with Dunn's post-test (* P<0.05, ** P<0.01). The red asterisk indicates a comparison with MyoWT. D) Sera from mice immunized with MLA092 and MLA025 Myomedins were tested for reactivity with RHPA-pseudotyped virus coated on ELISA panel in competition with 10E8 or VRC01.

FIG. 5. Neutralization titration of sera from MLA-immunized mice. Neutralization assays were performed using a set of HIV-1 Clade A, B, C, AE, and D pseudoviruses of Tier 2 or 3 with TZM-bl indicator cells. Serially diluted serum samples in duplicates were incubated with pseudoviruses. After the incubation pseudovirus with serum, TZM-bl cells at a density of 105 cells/ml were added, incubated, lysed, and after addition of substrate, luminescence was measured.

FIG. 6. An unrooted phylogenetic tree of 194 representative sequences from HIV-1 2019 Compendium of HIV-1 ENV genes (https://www.hiv.lan1.gov) enriched for 22 HIV-1 ENV sequences used in this analysis. HIV-1 strains are classified into four groups M, N, O and P. The major group M is further divided into nine genetically distinct subtypes A, B, C, D, AE, G, H, J, F and K. Each subtype contains hybrid viruses—circulating recombinant forms as a result of genetic material combination. For better orientation, only the 22 HIV-1 ENV sequences used in the analysis are visualized.

FIG. 7. Binding of MLB protein variants to PGT126 bNAb, IgG isotype control and BSA. Three selected MLB clones were assayed in ELISA. Each point is shown as the mean value of duplicate with standard deviation.

FIG. 8. Binding of MLD protein variants to PGT121 bNAb, IgG isotype control and BSA. Three selected MLD clones were assayed in ELISA. Each point is shown as the mean value of duplicate with standard deviation.

FIG. 9. Competition of MLB and MLD protein variants with HIV envelope glycoprotein gp120 for binding to PGT126 or PGT121 bNAb in ELISA. Their binding to PGT121 or PGT126 bnAb, respectively, was visualized by the anti-Flag M2 Ab-HRP conjugate or in the case of biotinylated protein by streptavidin-HRP. Each point is shown as the mean value of triplicates with standard deviation.

FIG. 10. Immunization of experimental mice with a panel of MLB variants induced serum antibodies specifically recognizing HIV-1 Env. Sera were collected and tested in their reactivity with multimerizing HIV-1 gp120 clade B consensus coated on ELISA plates. Antibody titers of IgG isotype were measured in ELISA. Statistical comparison was performed by ANOVA Kruskal-Wallis test with Dunn's post-test (* P<0.05).

FIG. 11. Binding of sera from mice immunized with MLB variants to HIV-1 multimeric gp120 is specifically inhibited by PGT126.

### EXAMPLES OF CARRYING OUT THE INVENTION

In the following text, the polypeptides of the invention, derived from human Myomesin-1 domain 10, are called Myomedins.

Materials and Methods

Production of Human Myomesin-1 Domain 10

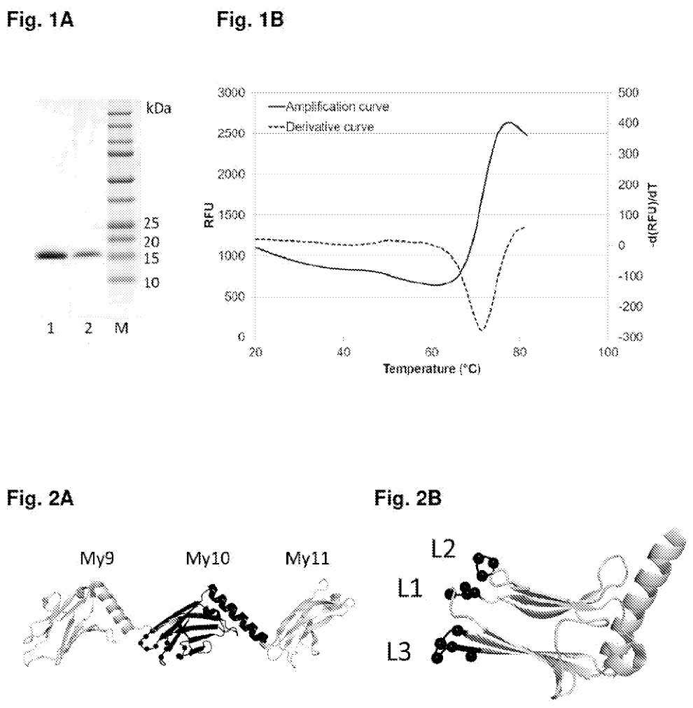

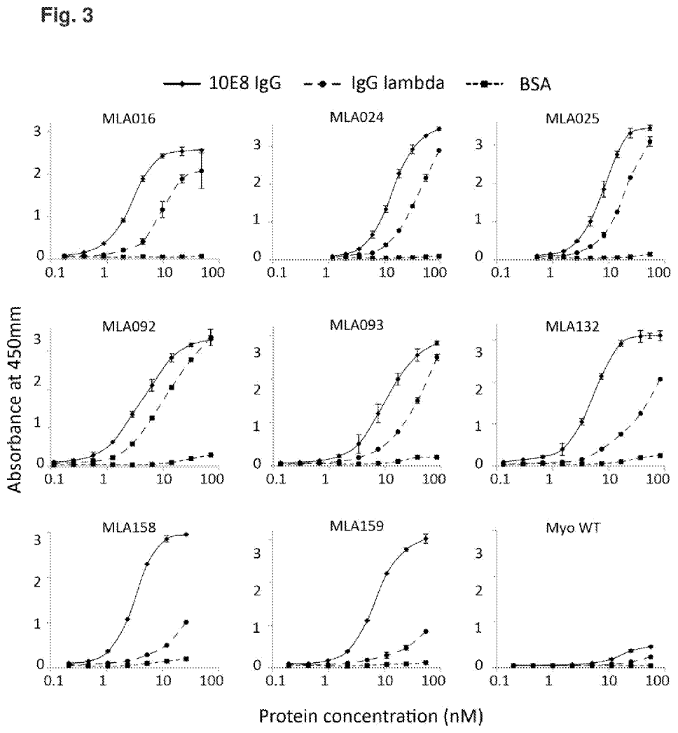

We produced domain 10 of myomesin-1 protein (MYOM1) in E. coli with N-terminal 6×His-tag and thrombin cleavage site. The protein was purified by Ni-NTA agarose and size-exclusion chromatography and its X-ray structure was solved to 1.8 Å resolution. The identified structure was deposited to the Protein Data Bank under accession code (PDB ID) 6t3o. Template DNA corresponding to the GenBank BC116183 was obtained from the Source BioScience (Nottingham, UK). Myomesin domain 10 was amplified by pair of primers (forward Myom10-F CATATGAAATCAGAGTTGGCAGTTGAAAT, SEQ ID NO. 17, reverse Myom10-R CAAGAATGGATCAGGAAACAAGGTTAAGGATCC, SEQ ID NO. 18) inserting NdeI and BamHI restriction sites that were then used for the construct ligation into the pET28b vector. The final protein product contains a 6×His-tag and thrombin cleavage site at the N-terminus. Myomesin was produced in E. coli BL21 (QDE3) cells. Cells grew at 37° C. to OD600 of 0.6 in LB (Lysogeny Broth) then the expression of the protein was induced by 1 mM IPTG and cultivation continued for 4 hours more before the cells were harvested by centrifugation. The protein was purified using Ni-NTA agarose (Qiagen, Germany) under native conditions. Eluted protein fractions were pooled, concentrated and applied to the size-exclusion chromatography column Superdex 75 10/300 GL (GE Healthcare, UK) with running buffer 20 mM Tris, 50 mM NaCl pH 8.0. As documented in FIG. 1A, produced protein analyzed by SDS-PAGE gel electrophoresis corresponds to an expected molecular weight 16 kDa.

Thermal Shift Assay

Protein samples (0.1 mg/ml) and 5× Sypro Orange dye (Sigma Aldrich) were added into total volume 25 μl. Using the real-time PCR Detection System CFX96 Touch (Bio-Rad Laboratories), the proteins were incubated in a thermal gradient from 20° C. to 80° C. at increments of 0.5° C. and with 30 s-hold intervals.

The degree of protein unfolding was monitored by FRET (fluorescence resonance energy transfer) channel that captured the spectral properties of Sypro Orange unfolded protein complexes (excitation wavelength≈470 nm and emission wavelength≈570 nm). The data were analyzed by CFX Manager software and the melting temperatures were determined using the first derivative spectra. As shown in FIG. 1B, identified temperature melting point (Tm) corresponds to Tm 72° C. This demonstrates that myomesin-1 domain 10 maintains structure conformation and stability even when it is separated from the structural context of the myomesin filament.

Design of Combinatorial Library Based on Scaffold of Human Myomesin-1 Domain

The crystal structure of the myomesin-1 domain 10 (PDB ID 6t3o) is formed by 111 amino acids, which constitute the 2-layer sandwich of the immunoglobulin-like domain with 7 antiparallel D-strands and a terminal a-helix (FIG. 2A, B). The domain corresponds to the MY10 part of the myosin filament-linking protein myomesin-1 PDB ID 2y23 with 100% identity. To select a set of myomesin residues suitable for randomization we have performed in silico mutability screening of all amino acid residues. We have used the crystal structure of human myomesin-1 domain 10 (PDB ID 6t3o) and mutated all residues to all 20 amino acids using the PositionScan routine of FoldX program. According to the predicted change of free energy upon mutation, we have assigned to each residue a mutability score corresponding to several stabilizing substitutions. The score was used for the final selection of candidate residues trying to find a continuous patch of accessible surface residues with high mutability. This procedure led to a selection of following residues localized in loop regions of myomesin domain 10: E1267, K1268, L1269, S1270, R1296, N1297, T1298, D1322, G1323, K1324, A1325, and T1326, numbered according to the UniProt P52179 record. The residues selected for randomization are shown in FIG. 2B.

Myomedin Library Construction

Myomedin combinatorial library was assembled by a series of three PCR using Phusion High-Fidelity DNA Polymerase (NEB, Massachusetts, USA) using a list of primers and adaptors (see Table 1. below). 1st PCR (annealing temperature 65° C., 10 cycles) with 100 μM oligonucleotides MYOM-LP_n1F, MYOM-LP_2F and MYOM-LP_n2R resulted in 147 bp product, which was used in 2nd PCR (annealing temperature 59° C., 10 cycles) with 10 μM oligonucleotides MYOM-LP_1F and MYOM-LP_3R. 3rd PCR with 10 μM oligonucleotides L-for and L-rev was used to complete Myomedin sequence (333 bp). Furthermore, two additional PCR was used, 4th PCR with primers JOIN-F (adding ribosome binding site RBS), and JOIN-R and finally 5th PCR with primers T7B (adding T7 promoter) and TolAk joining Myomedin and TolA spacer (E. coli str. K-12). TolA spacer template for the 5th PCR was amplified using primers P7 link and TolA rev from isolated genomic DNA of E. coli. The resulted PCR product representing linear vector for in vitro transcription and translation (5′-T7 promotor-5′stem loop-RBS-Myomedin-TolAk-3′stem loop-3′) was extracted from agarose gel and purified.

TABLE 1
List of oligonucleotides for combinatorial
library assembly and ribosome display.
Trinucleotide mixture of 19 amino acid
codons (Ella Biotech GmbH, Martinsried,
Germany) was used for the synthesis of
randomized positions (bold) of a coding
DNA strand (X01) and the complementary
DNA strand (R01).

| | |
|---|---|
| MYOM-LP_n1F | AATGCCAAAGTGAACTATAT |
| | CTTCAACGAGAAAGAAATCT |
| | TCGAAGGTCCGAAATACAAAA |
| | TGCATATTG |
| | (SEQ ID NO. 19) |
| MYOM-LP_2F | GGTCCGAAATACAAAATGCA |
| | TATTGATX01X01X01GGCA |
| | TCATCGAAATGTTTATGG |
| | (SEQ ID NO. 20) |
| MYOM-LP_n2R | CAGCTGAAAGGTATAGGTGC |
| | CTTCATCTTCATCCTGCAGT |
| | TTTTCCATAAACATTTCGAT |
| | GATGCC |
| | (SEQ ID NO. 21) |
| MYOM-LP_1F | TCGTTTTTGGATGCAGGCAX |
| | 01X01X01X01GGTAATGCC |
| | AAAGTGAACTATATCTTC |
| | (SEQ ID NO. 22) |
| MYOM-LP_3R | CCAGAACAACGGTTGAATGA |
| | TTR01R01R01R01R01CTG |
| | CAGCTGAAAGGTATAGGTGC |
| | (SEQ ID NO. 23) |
| L-for | AAAAGCGAGCTGGCCGTGGA |
| | AATTCTGGAAAAAGGTCAGG |
| | TTCGTTTTTGGATGCAGGCA |
| | (SEQ ID NO. 24) |
| L-rev | ACCCTGTTTACGAATCCATT |
| | CTTGGCGCTGAAATTCTGCT |
| | TCTTTTTGCAGTTTTTTGAA |
| | CACGTCACCAACCAGAACAA |
| | CGGTTGAATGATT |
| | (SEQ ID NO. 25) |
| JOIN-F | CTATAGGGAGACCACAACGG |
| | TTTCCCTCTAGAAATAATTT |
| | TGTTTAACTTTAAGAAGGAG |
| | ATATACATATGAAAAGCGAG |
| | CTGGCCG |
| | (SEQ ID NO. 26) |
| JOIN-R | GAACCGACCGCGGATCCACC |
| | CTGTTTACGAATCCATTCTT |
| | (SEQ ID NO. 27) |
| T7B | ATACGAAATTAATACGACTC |
| | ACTATAGGGAGACCACAACG |
| | G (SEQ ID NO. 28) |
| TolAk | CCGCACACCAGTAAGGTGTG |
| | CGGTTTCAGTTGCCGCTTTC |
| | TTTCT |
| | (SEQ ID NO. 29) |
| P7 link | GGATCCGCGGTCGGTTCGA |
| | (SEQ ID NO. 30) |
| TolA rev | TTTCCGCTCGAGCTACGGTT |
| | TGAAGTCCAATGGCGC |
| | (SEQ ID NO. 31) |
| His-Myo-F | CAGTCCATGGGCAGCAGCCA |
| | TCATCATCATCATCACAGCA |
| | GCGGCAAAAGCGAGCTGGCC |
| | G (SEQ ID NO. 32) |

Antibodies Used for Selection.

Broadly neutralizing human anti-HIV-1 Env monoclonal antibodies 10E8, PGT121, and PGT126 were obtained from the NIH AIDS Reagent Program (Division of AIDS, NIAID, NIH, Germantown, MD). 10E8, PGT121, or PGT126 IgG were used as a target protein for ribosome display and in ELISA applications (stored as 1 mg/mL aliquoted source stock at −80° C.). Human IgG1 with κ or λ light chain (light chain was used as complementary to tested bNAb, purified myeloma protein, Sigma-Aldrich, St. Luis, MO) was chosen as an isotype control for library preselection in ribosome display and as a negative control in ELISA (stored aliquoted as 1 mg/mL source stock at −20° C.).

Ribosome Display Selection

Myomedin combinatorial library was used for in vitro transcription/translation and further ribosome display (RD) selection. Three-round RD selections were performed, 96-well Polysorp plates (NUNC, Denmark) were coated by 10E8 IgG1 diluted in coating 100 mM bicarbonate/carbonate solution (pH 9.6) at a concentration according to the adjusted stringency in each round of RD selection procedure: 1st round—25 μg/mL, 2nd round—10 μg/mL and 3rd round—10 μg/mL. Pre-selection procedure was performed in wells coated with human IgG1 lambda antibody at a constant concentration of 25 μg/mL in each round. Final cDNA after the third round of the selection was amplified by PCR with primers His-Myo-F and JOIN-R. Cleaved PCR product (NcoI, BamHI) was introduced into a pET-28b vector carrying V5 tag sequence downstream of Myomedin cDNA and cloned in E. coli XL1 blue host cells. The same method was used to select proteins binding to PGT121 and PGT126 IgG.

Production of Myomedin Variants

Myomedin protein variants designated MLA, MLB and MLD (see Table 2.) were produced as 16 kDa recombinant proteins with N-terminal His6 tag and C-terminal V5 tag (His6-Myomedin-V5) in E. coli BL21 (DE3) strain in LB medium containing kanamycin (60 μg/mL). Protein production was induced by 1 mM IPTG after the culture reached the optical density OD600=0.6, cells were further cultivated, shaken at 250 RPM, 25° C. for 4 hours after induction. Harvested cells were sonicated in TN buffer (50 mM Tris, 150 mM NaCl, pH 8.0), centrifuged (40 000×g, 20 min. 4° C.) and subsequently, bacterial lysates were analyzed or protein was purified on Ni-NTA agarose column.

TABLE 2
Table of MLA, MLB and MLD sequences. Sequence
comparison of the protein variants with
parental non-randomized MyoWT protein. Grey
boxes indicate the loop stretches with 12
positions at which the residues of MyoWT
were randomized. Other residues of the
111 amino acid Myomedin scaffold protein
were not mutated in all shown binding proteins.

| | L1 | | | | | L2 | | | | L3 | | | | | |
|---|---|---|---|---|---|---|---|---|---|---|---|---|---|---|---|
| | 21 | 22 | 23 | 24 | | 50 | 51 | 52 | | 76 | 77 | 78 | 79 | 80 | SEQ ID NO. |
| MyoWT | E | K | L | S | . . . | R | N | T | | D | G | K | A | T | 1, 59, 60 |
| MLA016 | K | A | Q | Q | . . . | I | M | F | . . . | S | H | H | L | G | 3, 33, 47 |
| MLA024 | L | S | V | F | . . . | R | N | T | . . . | F | M | L | M | M | 4, 34, 48 |
| MLA025 | A | T | P | S. | . . . | G | H | E | . . . | V | I | L | I | L | 5, 35, 49 |
| MLA092 | E | I | M | W | . . . | P | S | W | . . . | I | V | T | P | L | 6, 36, 50 |
| MLA093 | D | G | S | S | . . . | R | A | N | . . . | D | F | I | I | W | 7, 37, 51 |
| MLA132 | L | L | P | L | . . . | Y | F | W | . . . | M | W | S | E | — | 8, 38, 52 |
| MLA158 | 3 | M | N | W | . . . | I | T | L | . . . | L | Y | Y | A | W | 9, 39, 53 |
| MLA159 | M | N | L | Y | . . . | Q | A | M | . . . | M | M | I | E | Y | 10, 40, 54 |
| MLB036 | M | W | R | N | . . . | R | N | T | . . . | W | M | T | Q | T | 11, 41, 55 |
| MLB041 | I | M | M | E | . . . | D | M | R | . . . | I | V | T | P | L | 12, 42, 50 |
| MLB049 | K | H | Q | L | . . . | W | L | W | . . . | I | V | T | P | L | 13, 43, 50 |
| MLD033 | H | W | Q | F | . . . | Q | G | E | . . . | P | Q | L | W | L | 14, 44, 56 |
| MLD068 | Y | A | G | N | . . . | V | Q | Y | . . . | E | P | I | F | L | 15, 45, 57 |
| MLD108 | H | G | Q | W | . . . | V | S | L | . . . | Q | T | A | T | Y | 16, 46, 58 |

ELISA

Cell lysates of E. coli clones producing Myomedin protein variants were prepared using sonicator (Misonix 3000). Polysorp plate (NUNC) was coated with 10E8, PGT121 or PGT126 IgG (5 μg/mL) or IgG1 lambda (5 μg/mL) in coating buffer (100 mM bicarbonate/carbonate solution, pH 9.6) at 7° C. overnight. Next day, the plate was washed by PBST solution (PBS buffer containing 0.05% Tween, pH 7.4) and wells were blocked by PBSTB (PBS buffer pH 7.4, containing 0.05% Tween and 1% BSA). The lysate samples (diluted 33×) purified protein variants as well as Myomedin-wt negative control diluted in PBSTB were applied. Binding of the Myomedin variants was detected using anti-V5 tag-HRP conjugate in PBSTB (1:10 000, Abcam, Cambridge, UK). Results were visualized by the enzymatic reaction of HRP with TMB-Complete 2 substrate (TestLine Clinical Diagnostics s.r.o., Brno, Czech Republic), the reaction was stopped by 2 M sulfuric acid and absorbance at 450 nm was measured.

Myomedin Variants Induce Serum Antibodies Recognizing Env Glycoproteins on Pseudotyped HIV-1 Viruses.

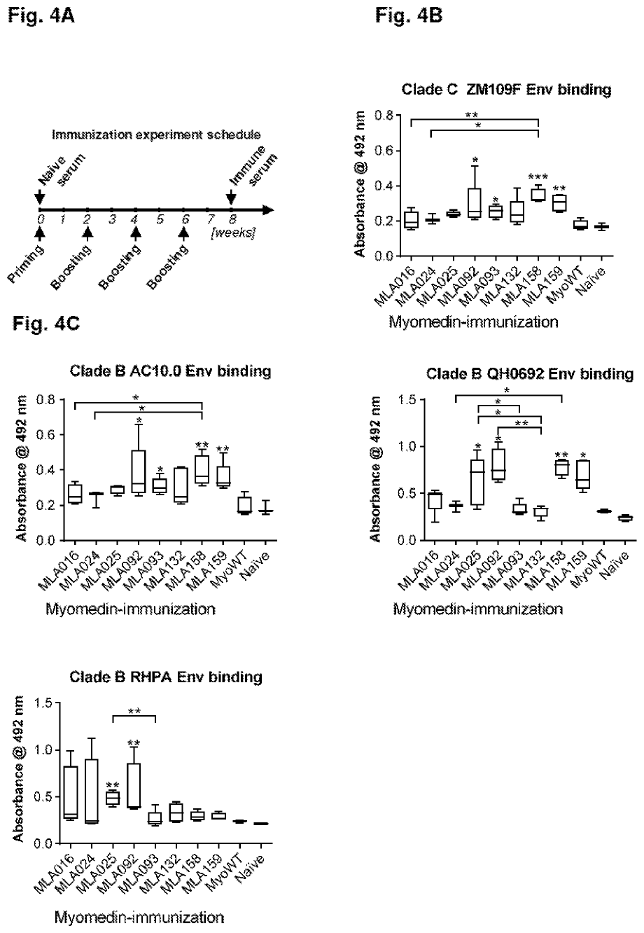

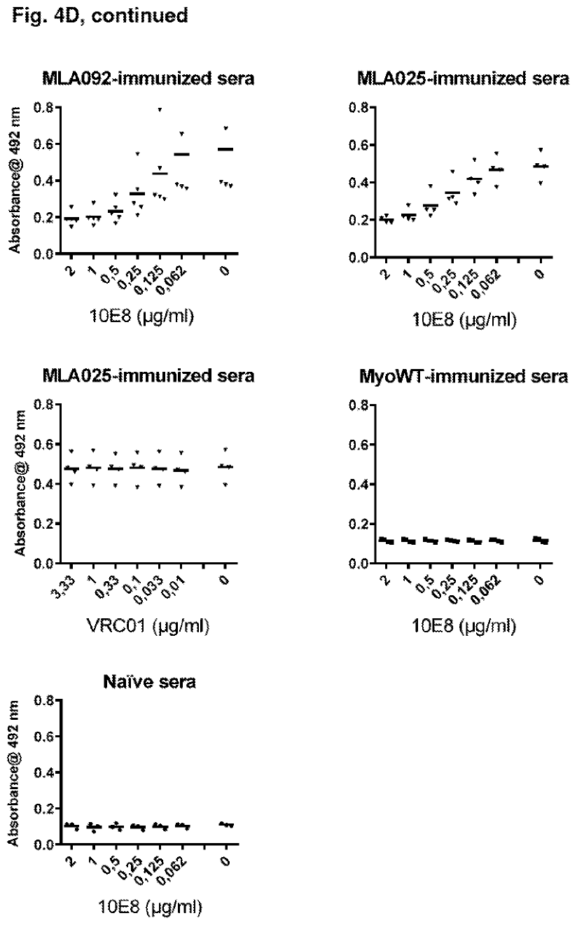

Eight MLA protein variants 016, 024, 025, 092, 093, 132, 158 and 159 were expressed and purified as recombinant proteins and used for immunization of experimental mice by i.d. route with Freund's adjuvant by four consecutive antigen injections, according to the schedule presented in FIG. 4A. MPER-specific reaction was evaluated on pseudoviruses coated on ELISA panel as the presence of membrane surrounding the gp41 is necessary to MPER epitope. Four pseudoviruses—one Clade C Env (ZM109F) (FIG. 4B) and three Clade B Env (AC10.0, QH0692, RHPA) (FIG. 4C) were assayed on ELISA. The experiment identified significant binding for MLA 025, 092, 093, 158 and 159 at least with one pseudotyped virus (FIG. 4B, C) when compared with wild-type Myomedin (MyoWT). MyoWT did not elicit detectable Env-pseudovirus specific serum response. To further confirm MPER specificity of sera binding, we performed an ELISA competition assay where the binding of sera to selected pseudovirus from Clade B (here RHPA) competed with a serial dilution of 10E8 as an original specific target used for all MLA variants selection (FIG. 4D). 10E8 in concentration 2 μg/ml completely inhibited all tested hyperimmune sera reactivity. When irrelevant bNAb was used for competition (here VRC01) no competition was observed even at concentration 3.33 μg/ml. As negative controls, naive sera and Myomedin wild type-immunized mice sera were tested in the same assay and no binding of sera to RHPA was observed irrespective to titration of 10E8 as a competitor (FIG. 4D). 10E8 serially diluted to achieve final concentrations 2; 1; 0.5; 0.25; 0.125; 0.0625; and 0 μg/ml in blocking buffer was applied with individual MLA092- or MLA025-immunized mouse sera diluted 1:400. After washing, the plates were incubated with rabbit anti-mouse IgG HRP-conjugated antibody, developed with a substrate and O.D. 492 nm was measured. VRC01 antibody was applied in control reaction, as irrelevant antibody, at final 3.33; 1; 0.33; 0.1; 0.033; and 0 μg/ml analogously to 10E8. In separate experiment 10E8 at indicated concentration was applied with individual MyoWT-immunized or naive mouse sera diluted 1:400 as the control. All experiments were performed in triplicates. Mean values are indicated by horizontal lines.

Immunization of Experimental Mice

Female BALB/c mice, 6-8 weeks old, 18-22 g (AnLab, Brno, Czech Republic), were used for all immunization experiments. Animals were housed under standard conditions according to ARRIVE guidelines. The immunization experiments were approved by the Ethics Committee of the Faculty of Medicine and Dentistry (Palacky University, Olomouc, Czech Republic) and the Ministry of Education, Youth and Sports, Czech Republic (MSMT-9487/2019-3). All mice were immunized four times. Pre-immune (naïve) sera were collected before the first immunization. All immunizations were performed by intradermal route with an equal dose of 10 μg of individual Myomedin variant (diluted in 50 μl of DPBS) mixed with 50 μl of Freund's adjuvant (Sigma Aldrich, St. Louis, MO, USA) per mouse per one immunization.

Neutralization Titration of Sera from MLA-Immunized Mice.

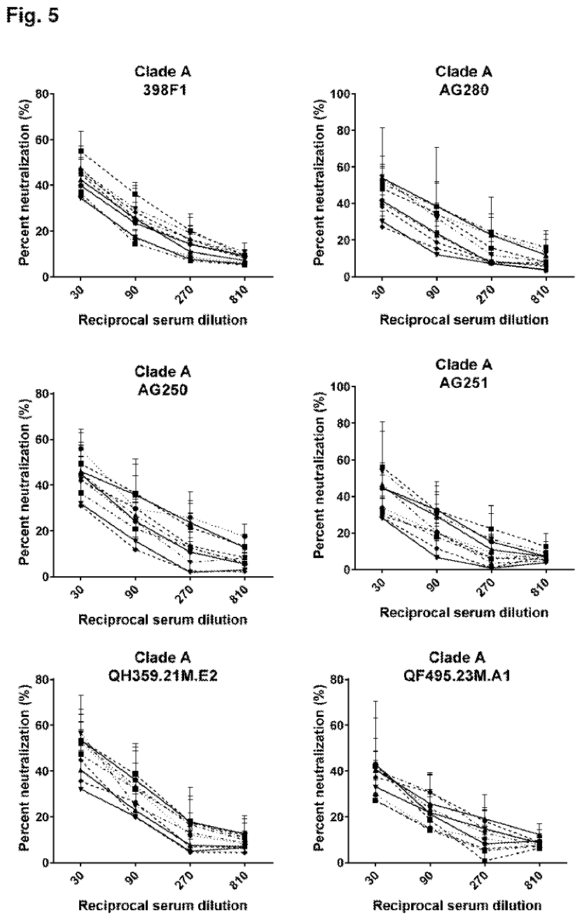

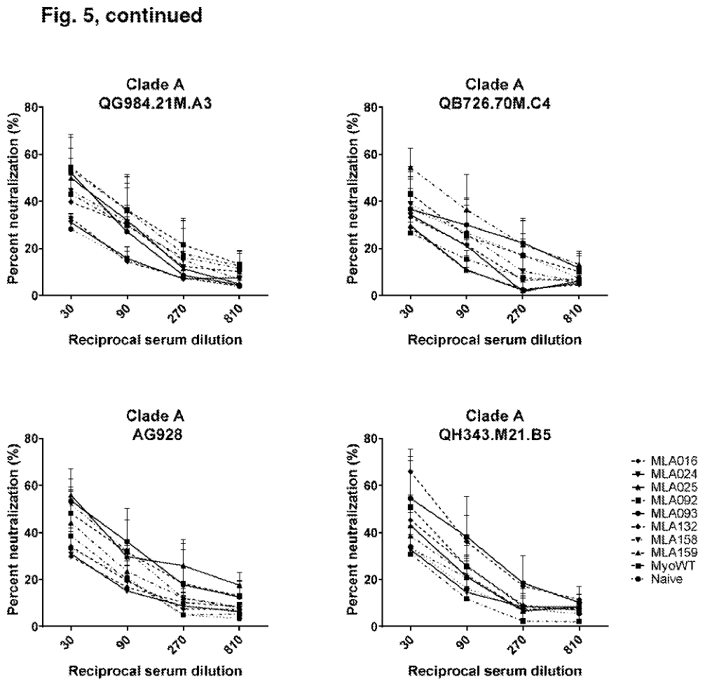

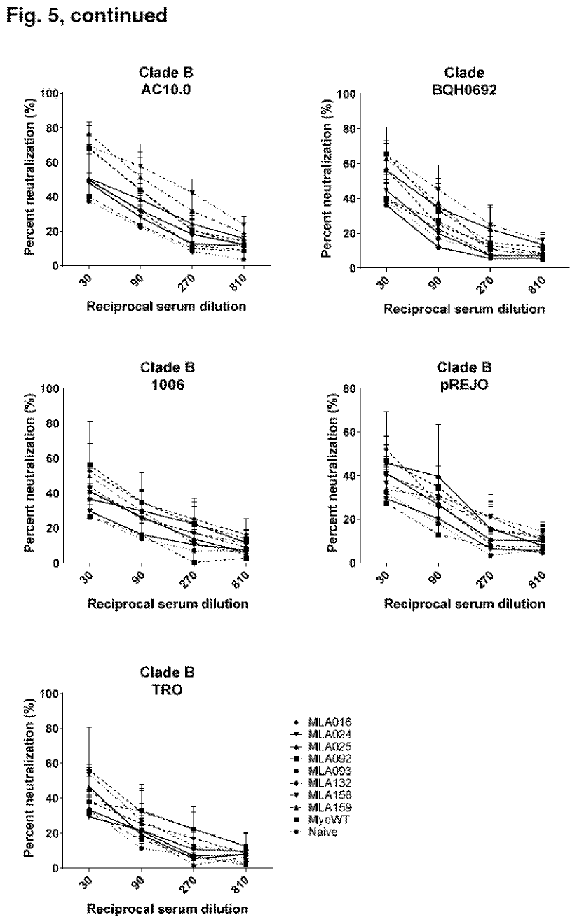

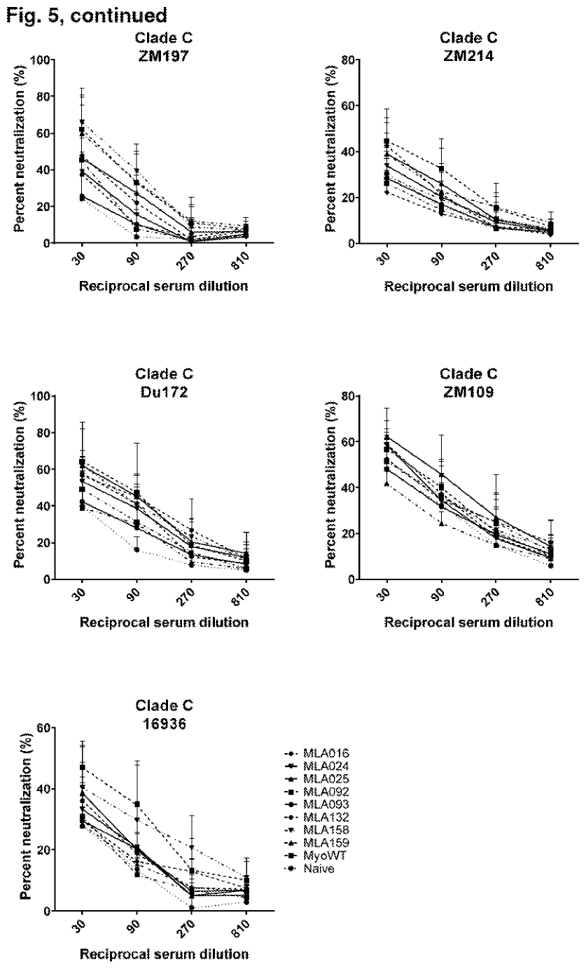

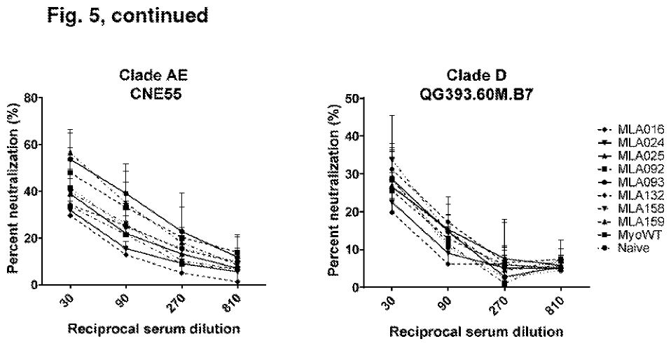

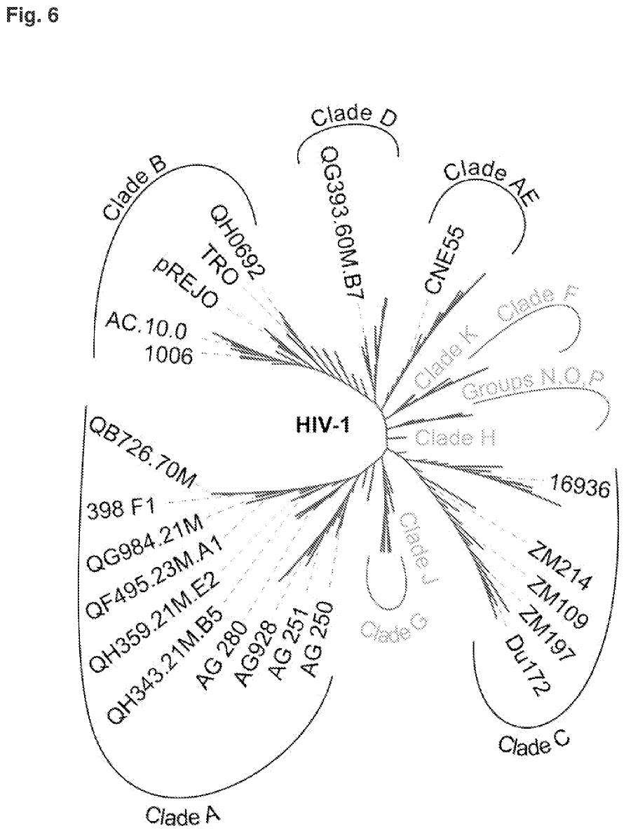

Mice were immunized by the administration of four doses of individual MLA variants including wild-type (MyoWT). Each group consisted of five animals. Neutralization assays were performed using a set of HIV-1 Clade A, B, C, AE, and D pseudoviruses of Tier 2 or 3 with TZM-bl indicator cells. Serially diluted serum samples in duplicates were incubated with pseudoviruses. The pseudoviruses load was set to achieve approximately 150 000 RLU in 150 μl of DMEM in the absence of sera. After the incubation pseudovirus with serum, TZM-bl cells at a density of 105 cells/ml were added, incubated, lysed, and after addition of substrate, luminescence was measured. Results are indicated in FIG. 5. The 22 HIV-1 ENV sequences used in the analysis are visualized in FIG. 6.

HIV-1 Env Specific Binding Antibodies Determination

Reactivity of binding antibodies targeting Env was measured by ELISA using pseudotyped viruses prepared for virus neutralization assay (MLA polypeptides see below) or recombinant HIV-1 consensus clade B multimerizing polypeptides (MLB and MLD). To remove fetal bovine serum pseudotyped viruses were first ultracentrifuged for 3 hours at 50.000 g at 4° C. and resuspended in PBS. MaxiSorp ELISA plates (NUNC, Roskilde, Denmark) were coated with pseudotyped viruses diluted in PBS overnight at 4° C. Coating with recombinant gp120 was performed under the same conditions. Plates were washed with PBS and blocked with 1% BSA/PBS for 3 hours at room temperature. Mouse sera were serially diluted in blocking buffer in duplicates and incubated overnight at 4° C. Plates were washed with PBS and bound antibodies targeting Env were determined by incubating with anti-mouse IgG secondary antibody conjugated with horseradish peroxidase diluted in blocking buffer for 3 hours. Plates were washed and the signal was developed with O-phenylenediamine-H₂O₂ substrate. The reaction was stopped with IM sulphuric acid. The absorbance was quantified at 492 nm by ELISA reader.

Competition ELISA

Plates were coated with the selected HIV-1 pseudotyped virus or recombinant HIV-1 multimerizing gp120 protein as described in the previous method of Env-specific binding antibodies determination. 10E8, PGT126, or PGT121 antibodies were serially diluted in blocking buffer and applied in doublets with mouse hyperimmune sera diluted accordingly. Plates were washed and bound mouse antibodies were detected by rabbit anti-mouse IgG secondary antibody conjugated with horseradish peroxidase diluted in blocking buffer. Signal was developed and measured as described above.

Virus Preparation

Pseudotyped viruses were prepared using HEK293/17 cell line grown in 75 cm2 at 60-80% confluency co-transfected with 4 μg of plasmid coding Env and 8 μg of plasmid coding pSG3deltaEnv mixed with 48 μl of transfection reagent FuGene6 (Promega, Madison, WI, USA) in 12 ml of culture medium. After 2 days, produced pseudotyped viruses in culture medium were harvested, filtered and stored at 80° C. until used.

Virus Neutralization Assay

Virus neutralization assay was done using various pseudotyped viruses of clades A, B, C, D and AE. Murine leukemia virus (MULV)-pseudotyped virus containing the Env of MULV with the same backbone vector as all HIV-1-pseudotyped viruses (pSG3AEnv) was used as a negative control. Before the assay, mouse sera were heat-inactivated for 1 hour at 56° C. Sera in duplicates were serially diluted in 100 μl of culture medium in 96-well plates and incubated with 50 μl of pseudotyped viruses at 150.000 RLU for 90 minutes at 37° C. Then, 10.000 TZM-bl cells stably expressing CD4 receptor, CCR5, CXCR4 co-receptors and containing genes for luciferase and β-galactosidase under control of HIV-1 promotor (NIH AIDS Research and Reference Reagent Program, Division of AIDS, NIAID, NIH) in 50 μl of culture medium were added into each well and incubated for 48 hours at 37° C. in 5% CO2 atmosphere. 150 μl of culture medium was removed and 100 μl of lysis buffer containing luciferin (Promega) was added. After 2 minutes, 100 μl of lysate was transferred into a black 96-well plate and luminescence was measured using HP luminometer. This method was used to measure neutralization activity of MLA immunized sera (Table 3.) and MLB immunized sera (Table 4.).

TABLE 3
Neutralization activity of MLA-immunized mice sera. Values represent the
reciprocal serum dilution which resulted in 50% virus neutralization. Relative
mean neutralization dil. represents mean values of detected titers among all
tested pseudoviruses. No. of neutralized PVs indicates the sum of pseudoviruses
neutralized at reciprocal titer higher than 30. Murine leukemia virus (MULV)
was used in neutralization assay as a control. Pseudotyped viruses used for
analyses were chosen to cover the phylogenetic tree of HIV-1 Env.
Reciprocal serum dilution resulted in 50% virus neutralization

| | | Myomedin MLA | | | | | | | | | |
|---|---|---|---|---|---|---|---|---|---|---|---|
| Clade | Pseudovirus | 016 | 024 | 025 | 092 | 093 | 132 | 158 | 159 | WT | Naïve |
| AE | CNE55 | < | < | 41 | < | < | 35 | < | < | < | < |
| A | 398F1 | < | < | < | 35 | < | < | < | < | < | < |
| | AG280 | 35 | 31 | < | 35 | < | < | < | 32 | < | < |
| | QG984.21M.A3 | < | < | 35 | < | < | 33 | 38 | < | < | < |
| | AG250 | 33 | < | < | < | < | < | 37 | < | < | < |
| | AG251 | < | < | < | 43 | < | < | 46 | < | < | < |
| | QB726.70M.C4 | < | < | 38 | < | < | < | < | < | < | < |
| | QH359.21M.E2 | 34 | < | 45 | 42 | < | 36 | 38 | < | < | < |
| | QF495.23M.A1 | < | < | < | < | < | < | < | < | < | < |
| | AG928 | < | < | < | 34 | < | 35 | < | 34 | < | < |
| | QH343.21M.B5 | < | < | < | 32 | 44 | 57 | < | < | < | < |
| B | AC.10.0 | < | < | 30 | 56 | < | 59 | 92 | 78 | < | < |
| | QH0692 | < | < | 45 | 57 | < | 40 | 75 | 53 | < | < |
| | pREJO | 33 | < | 60 | < | < | < | < | < | < | < |
| | 1006 | < | < | < | 42 | < | 35 | < | < | < | < |
| | TRO | 45 | < | < | < | < | < | 42 | < | < | < |
| C | ZM197 | < | < | < | 44 | < | < | 63 | 43 | < | < |
| | ZM214 | < | < | < | < | < | < | < | < | < | < |
| | ZM109 | 52 | 47 | 72 | 52 | < | 37 | 38 | < | < | < |
| | Du172 | 58 | 37 | 62 | 74 | < | 57 | 60 | < | < | < |
| | 16936 | < | < | < | < | < | < | < | < | < | < |
| D | QG393.60M.B7 | < | < | < | < | < | < | < | < | < | < |
| CTRL | MULV | < | < | < | < | < | < | < | < | < | < |

| | | | | | | | | |
|---|---|---|---|---|---|---|---|---|
| Relative mean | 41 | 38 | 47 | 45 | 44 | 42 | 52 | 48 |
| neutralization dil. | | | | | | | | |
| No. of neutralized PVs | 7 | 3 | 9 | 12 | 1 | 10 | 10 | 5 |

TABLE 4
Neutralization activity of MLB- and MLD-immunized mice sera. Values represent the
reciprocal serum dilution which resulted in 50% virus neutralization. Relative
mean neutralization dil. represents mean values of detected titers among all
tested pseudoviruses. No. of neutralized PVs indicates the sum of pseudoviruses
neutralized at reciprocal titer higher than 30. Murine leukemia virus (MULV)
was used in neutralization assay as a control. Pseudotyped viruses used for
analyses were chosen to cover the phylogenetic tree of HIV-1 Env.
Reciprocal serum dilution resulted in 50% virus neutralization

| | | MLB - PGT126 | | | MLD - PGT121 | | | | |
|---|---|---|---|---|---|---|---|---|---|
| Clade | Pseudovirus | 036 | 041 | 049 | 033 | 068 | 108 | WT | Naïve |
| AE | CNE55 | < | < | < | < | < | < | < | < |
| A | 398F1 | < | 35 | < | < | < | < | < | < |
| | AG280 | 35 | 42 | 38 | < | < | 39 | < | < |
| | QG984.21M.A3 | 31 | 33 | < | < | < | < | < | < |
| | AG250 | < | < | < | < | < | < | < | < |
| | AG251 | < | < | < | < | < | < | < | < |
| | QB726.70M.C4 | < | < | < | < | < | < | < | < |
| | QH359.21M.E2 | 37 | 45 | 33 | < | < | 35 | < | < |
| | QF495.23M.A1 | 43 | 49 | 45 | < | < | 38 | < | < |
| | AG928 | < | < | < | < | < | < | < | < |
| | QH343.21M.B5 | 38 | < | < | < | < | 32 | < | < |
| B | AC.10.0 | < | < | < | < | < | < | < | < |
| | QH0692 | < | 45 | 42 | < | < | 35 | < | < |
| | pREJO | 38 | 53 | < | < | < | 37 | < | < |
| | 6535 | 41 | < | 32 | < | < | < | < | < |
| | TRO | 31 | 35 | 34 | < | < | 34 | < | < |
| C | ZM197 | < | < | < | < | < | < | < | < |
| | ZM214 | < | < | < | < | < | < | < | < |
| | ZM109 | 33 | < | 32 | < | 33 | 38 | < | < |
| | Du172 | < | < | < | < | < | < | < | < |
| | 16936 | 51 | < | 33 | < | 31 | 35 | < | < |
| D | QG393.60M.B7 | < | < | < | < | < | < | < | < |
| CTRL | MULV | < | < | < | < | < | < | < | < |

| | | | | | | |
|---|---|---|---|---|---|---|
| Relative mean | 37.8 | 42.1 | 36.1 | 30 | 32 | 35.8 |
| neutralization dil. | | | | | | |
| No. of neutralized PVs | 10 | 8 | 8 | 0 | 2 | 9 |

Binding of MLB Protein Variants to PGT126 Broadly Neutralizing Antibody.

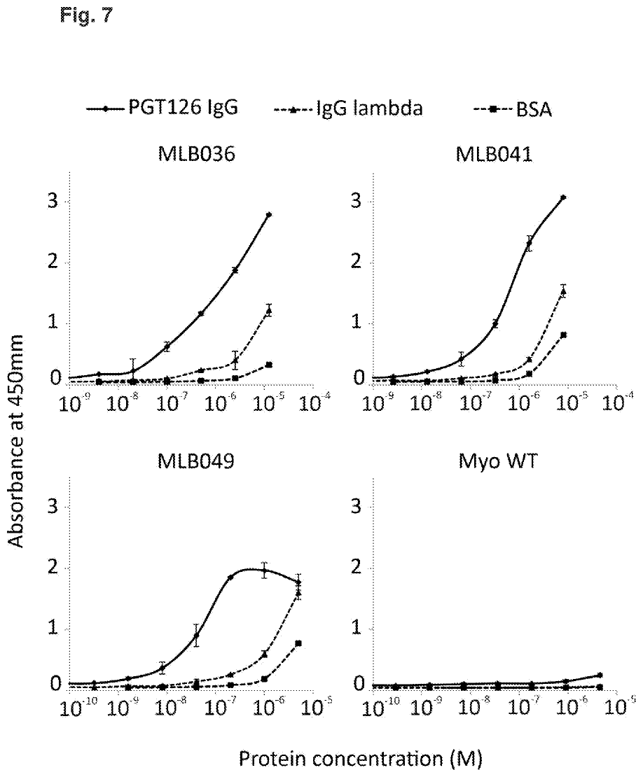

Three selected MLB clones 036, 041 and 049 in the form of purified fusion proteins with N-terminal polyhistidine tag and C-terminal V5 tag produced in E. coli BL21 (DE3) were assayed in ELISA, the parental non-randomized Myomedin was used as a negative control. Binding to immobilized PGT126 bNAb (labeled as PGT126 IgG), IgG1λ isotype (labeled as IgG lambda) and BSA was detected by anti-V5 Ab-HRP conjugate. Each point is shown as the mean value of duplicate with standard deviation. Results are presented in FIG. 7.

Binding of MLD Protein Variants to PGT121 Broadly Neutralizing Antibody.

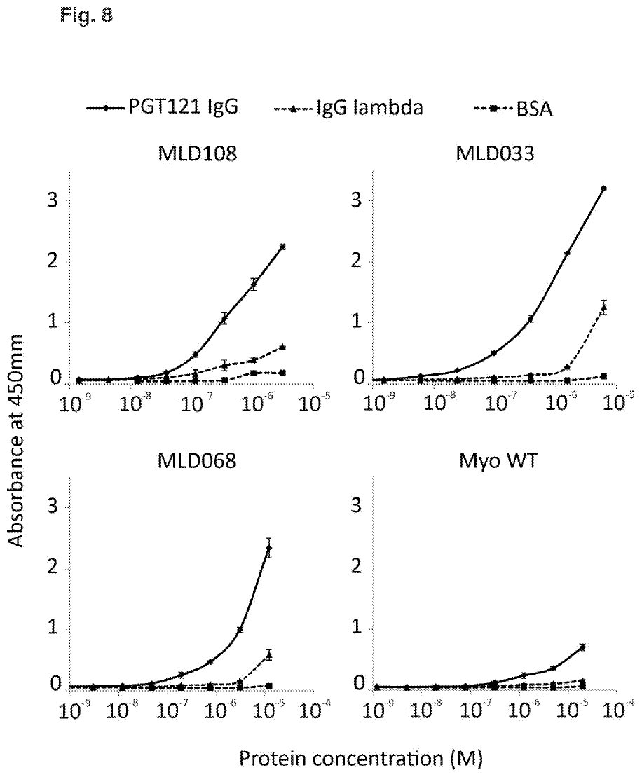

Selected MLD clones 108, 033 and 068 in the form of purified fusion proteins with N-terminal polyhistidine tag and C-terminal V5 tag produced in E. coli BL21 (DE3) were assayed in ELISA, the parental non-randomized Myomedin was used as a negative control. Binding to immobilized PGT121 bNAb (labeled as PGT121 IgG), IgGli isotype (labeled as IgG lambda) and BSA was detected by anti-V5 Ab-HRP conjugate. Each point is shown as the mean value of duplicate with standard deviation. Results are demonstrated in FIG. 8.

Competition of MLB and MLD Protein Variants with HIV Envelope Glycoprotein Gp120 for Binding to PGT126 or PGT121 bNAb in ELISA.

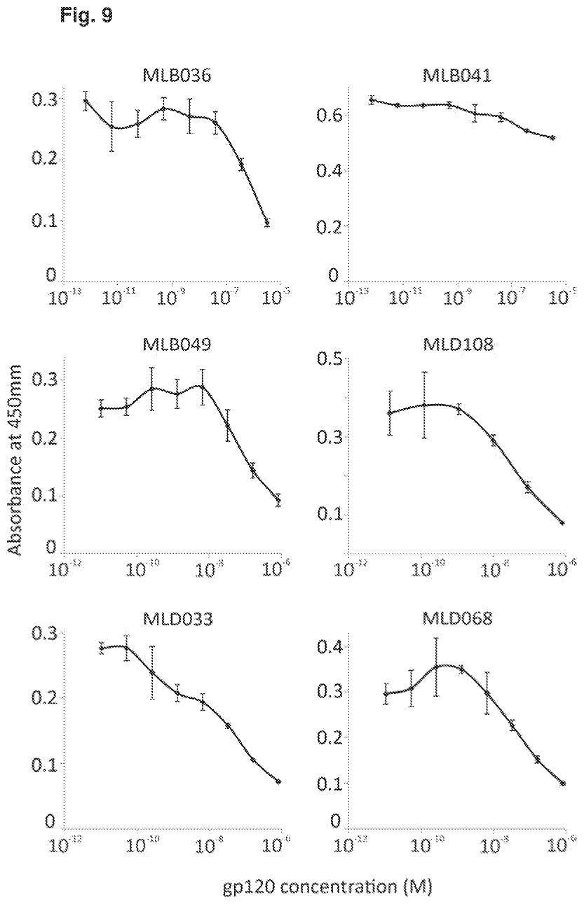

The protein variants MLB036, MLB041, MLD033, MLD108 (His6-Myo-Flag) were produced in E. coli BL21 and in vivo biotinylated variants MLD068 and MLD049 (His6-Myo-Avi) in E. coli BL21 BirA strain. Proteins were purified using Ni-NTA agarose and assayed in ELISA. Increasing concentration of gp120 inhibits binding of the MLB and MLD proteins at a constant concentration μM to bnAb. Their binding to PGT121 or PGT126 bnAb, respectively, was visualized by the anti-Flag M2 Ab-HRP conjugate or in the case of biotinylated protein by streptavidin-HRP. Each point is shown as the mean value of triplicates with standard deviation. Results are presented in FIG. 9.

Immunization of Experimental Mice with a Panel of MLB Variants Induced Serum Antibodies Specifically Recognizing HIV-1 Env.

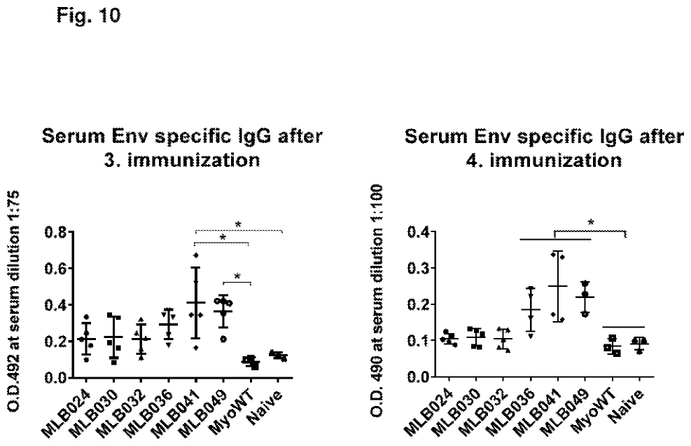

Mice were immunized by the administration of four doses of individual MLB variants including wild-type (MyoWT). Following immunization, sera were collected and tested in their reactivity with multimerizing HIV-1 gp120 clade B consensus coated on ELISA plates. Antibody titers of IgG isotype were measured in ELISA. Statistical comparison was performed by ANOVA Kruskal-Wallis test with Dunn's post-test (* P<0.05). Results are shown in FIG. 10.

Binding of Sera from Mice Immunized with MLB Variants to HIV-1 Multimeric Gp120 is Specifically Inhibited by PGT126.

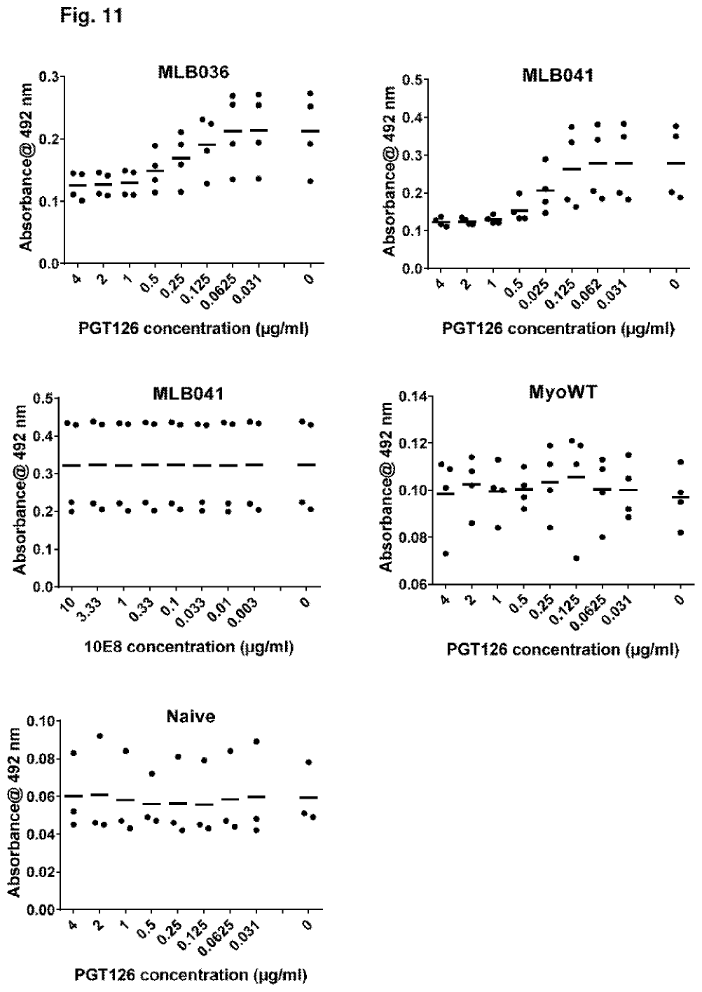

Sera from mice immunized with MLB036 and MLB041 were tested for reactivity with recombinant multimeric HIV-1 clade B consensus protein coated on ELISA panel in competition with PGT126 as specific inhibitor of MLB or 10E8 (irrelevant mAb control) serially diluted to achieve final concentrations as indicated in blocking buffer was applied with individual MLB036- or MLB041-immunized mouse sera diluted 1:75 after 3rd or 1:200 after 4th immunization. After washing, plates were incubated with rabbit anti-mouse IgG HRP-mAb, developed with a substrate and O.D. 492 nm was measured. 10E8 was applied in control reaction at indicated concentrations. In separate experiment PGT126 at indicated concentration was applied with individual MyoWT-immunized or naive mouse sera diluted 1:200 as the control (MyoWT). Mean values are indicated by horizontal lines. Results are presented in FIG. 11.

1. A polypeptide having a length of up to 180 amino acids and containing a sequence selected from sequences identical or differing at most in 5 amino acids from the sequence:
(SEQ ID NO. 2)
KSELAVEILEKGQVRFWMQAX₂₁X₂₂X₂₃X₂₄GNAKVNY
IFNEKEIFEGPKYKMHIDX₅₀X₅₁X₅₂GIIEMFMEKLQD
EDEGTYTFQLQX₇₆X₇₇X₇₈X₇₉X₈₀NHSTVVLVGDVFK
KLQKEAEFQRQEWIRKQG,
wherein
X₂₁X₂₂X₂₃X₂₄ is selected from KAQQ (SEQ ID NO. 33), LSVF (SEQ ID NO. 34), ATPS (SEQ ID NO. 35), EIMW (SEQ ID NO. 36), DGSS (SEQ ID NO. 37), LLPL (SEQ ID NO. 38), WMWW (SEQ ID NO. 39), MNLY (SEQ ID NO. 40), MWRN (SEQ ID NO. 41), IMME (SEQ ID NO. 42), KHQL (SEQ ID NO. 43), HWQF (SEQ ID NO. 44), YAGN (SEQ ID NO. 45) and HGQW (SEQ ID NO. 46);
X₅₀X₅₁X₅₂ is selected from RNT, IMF, GHE, PSW, RAN, YFW, ITL, QAM, DMR, WLW, QGE, VQY and VSL;
X₇₆X₇₇X₇₈X₇₉X₈₀ is selected from SHHLG (SEQ ID NO. 47), FMLMM (SEQ ID NO. 48), VILIL (SEQ ID NO. 49), IVTPL (SEQ ID NO. 50), DFIIW (SEQ ID NO. 51), MWSE (deletion) (SEQ ID NO. 52), LYYAW (SEQ ID NO. 53), MMIEY (SEQ ID NO. 54), WMTQT (SEQ ID NO. 55), PQLWL (SEQ ID NO. 56), EPIFL (SEQ ID NO. 57) and QTATY (SEQ ID NO. 58);
said polypeptide optionally further having an affinity tag attached to the N-terminus or to the C-terminus of the sequence.

2. The polypeptide according to claim 1, which has the length of up to 180 amino acids and contains a sequence selected from sequences identical or differing at most in 5 amino acids from the sequences:
(SEQ ID NO. 3)
KSELAVEILEKGQVRFWMQAKAQQGNAKVNYIFNEKEIFE
GPKYKMHIDIMFGIIEMFMEKLQDEDEGTYTFQLQSHHLG
NHSTVVLVGDVFKKLQKEAEFQRQEWIRKQG
(SEQ ID NO. 4)
KSELAVEILEKGQVRFWMQALSVFGNAKVNYIFNEKEIFE
GPKYKMHIDRNTGIIEMFMEKLQDEDEGTYTFQLQFMLMM
NHSTVVLVGDVFKKLQKEAEFQRQEWIRKQG
(SEQ ID NO. 5)
KSELAVEILEKGQVRFWMQAATPSGNAKVNYIFNEKEIFE
GPKYKMHIDGHEGIIEMFMEKLQDEDEGTYTFQLQVILIL
NHSTVVLVGDVFKKLQKEAEFQRQEWIRKQG
(SEQ ID NO. 6)
KSELAVEILEKGQVRFWMQAEIMWGNAKVNYIFNEKEIFE
GPKYKMHIDPSWGIIEMFMEKLQDEDEGTYTFQLQIVTPL
NHSTVVLVGDVFKKLQKEAEFQRQEWIRKQG
(SEQ ID NO. 7)
KSELAVEILEKGQVRFWMQADGSSGNAKVNYIFNEKEIFE
GPKYKMHIDRANGIIEMFMEKLQDEDEGTYTFQLQDFIIW
NHSTVVLVGDVFKKLQKEAEFQRQEWIRKQG
(SEQ ID NO. 8)
KSELAVEILEKGQVRFWMQALLPLGNAKVNYIFNEKEIFE
GPKYKMHIDYFWGIIEMFMEKLQDEDEGTYTFQLQMWSEN
HSTVVLVGDVFKKLQKEAEFQRQEWIRKQG
(SEQ ID NO. 9)
KSELAVEILEKGQVRFWMQAWMWWGNAKVNYIFNEKEIFE
GPKYKMHIDITLGIIEMFMEKLQDEDEGTYTFQLQLYYAW
NHSTVVLVGDVFKKLQKEAEFQRQEWIRKQG
(SEQ ID NO. 10)
KSELAVEILEKGQVRFWMQAMNLYGNAKVNYIFNEKEIFE
GPKYKMHIDQAMGIIEMFMEKLQDEDEGTYTFQLQMMIEY
NHSTVVLVGDVFKKLQKEAEFQRQEWIRKQG
(SEQ ID NO. 11)
KSELAVEILEKGQVRFWMQAMWRNGNAKVNYIFNEKEIFE
GPKYKMHIDRNTGIIEMFMEKLQDEDEGTYTFQLQWMTQT
NHSTVVLVGDVFKKLQKEAEFQRQEWIRKQG
(SEQ ID NO. 12)
KSELAVEILEKGQVRFWMQAIMMEGNAKVNYIFNEKEIFE
GPKYKMHIDDMRGIIEMFMEKLQDEDEGTYTFQLQIVTPL
NHSTVVLVGDVFKKLQKEAEFQRQEWIRKQG
(SEQ ID NO. 13)
KSELAVEILEKGQVRFWMQAKHQLGNAKVNYIFNEKEIFE
GPKYKMHIDWLWGIIEMFMEKLQDEDEGTYTFQLQIVTPL
NHSTVVLVGDVFKKLQKEAEFQRQEWIRKQG
(SEQ ID NO. 14)
KSELAVEILEKGQVRFWMQAHWQFGNAKVNYIFNEKEIFE
GPKYKMHIDQGEGIIEMFMEKLQDEDEGTYTFQLQPQLWL
NHSTVVLVGDVFKKLQKEAEFQRQEWIRKQG
(SEQ ID NO. 15)
KSELAVEILEKGQVRFWMQAYAGNGNAKVNYIFNEKEIFE
GPKYKMHIDVQYGIIEMFMEKLQDEDEGTYTFQLQEPIFL
NHSTVVLVGDVFKKLQKEAEFQRQEWIRKQG
and
(SEQ ID NO. 16)
KSELAVEILEKGQVRFWMQAHGQWGNAKVNYIFNEKEIFE
GPKYKMHIDVSLGIIEMFMEKLQDEDEGTYTFQLQQTATY
NHSTVVLVGDVFKKLQKEAEFQRQEWIRKQG,
said polypeptide optionally further having an affinity tag attached to the N-terminus or to the C-terminus of the sequence.

3. The polypeptide according to claim 1, having the following variables in SEQ ID NO. 2:
X₂₁X₂₂X₂₃X₂₄ is selected from KAQQ (SEQ ID NO. 33), LSVF (SEQ ID NO. 34), ATPS (SEQ ID NO. 35), EIMW (SEQ ID NO. 36), DGSS (SEQ ID NO. 37), LLPL (SEQ ID NO. 38), WMWW (SEQ ID NO. 39), and MNLY (SEQ ID NO. 40);
X₅₀X₅₁X₅₂ is selected from RNT, IMF, GHE, PSW, RAN, YFW, ITL and QAM;
X₇₆X₇₇X₇₈X₇₉X₈o is selected from SHHLG (SEQ ID NO. 47), FMLMM (SEQ ID NO. 48), VILIL (SEQ ID NO. 49), IVTPL (SEQ ID NO. 50), DFIIW (SEQ ID NO. 51), MWSE (deletion) (SEQ ID NO. 52), LYYAW (SEQ ID NO. 53) and MMIEY (SEQ ID NO. 54).

4. The polypeptide according to claim 1, having the following variables in SEQ ID NO. 2:
X₂₁X₂₂X₂₃X₂₄ is selected from MWRN (SEQ ID NO. 41), IMME (SEQ ID NO. 42) and KHQL (SEQ ID NO. 43);
X₅₀X₅₁X₅₂ is selected from RNT, DMR and WLW;
X₇₆X₇₇X₇₈X₇₉X₈₀ is selected from IVTPL (SEQ ID NO. 50) and WMTQT (SEQ ID NO. 55).

5. The polypeptide according to claim 1, having the following variables in SEQ ID NO. 2:
X₂₁X₂₂X₂₃X₂₄ is selected from HWQF (SEQ ID NO. 44), YAGN (SEQ ID NO. 45) and HGQW (SEQ ID NO. 46);
X₅₀X₅₁X₅₂ is selected from QGE, VQY and VSL;
X₇₆X₇₇X₇₈X₇₉X₈₀ is selected from PQLWL (SEQ ID NO. 56), EPIFL (SEQ ID NO. 57) and QTATY (SEQ ID NO. 58).

6. The polypeptide according to claim 1, which has the length of up to 160 amino acids.

7. A conjugate of the polypeptide according to claim 1 with serum albumin or with heat shock protein hsp70.

8. The polypeptide according to claim 1 for induction of HIV-1 virus-neutralizing antibodies in a subject in need thereof.

9. The polypeptide according to claim 3 for induction of HIV-1 virus-neutralizing antibodies targeting the epitope targeted by broadly neutralizing antibody 10E8 in a subject in need thereof.

10. The polypeptide according to claim 4 for induction of HIV-1 virus-neutralizing antibodies targeting the epitope targeted by broadly neutralizing antibody PGT126 in a subject in need thereof.

11. The polypeptide according to claim 5 for induction of HIV-1 virus-neutralizing antibodies targeting the epitope targeted by broadly neutralizing antibody PGT121 in a subject in need thereof.

12. The polypeptide according to claim 1 for prevention of diseases or disorders caused by HIV-1 virus.

13. A vaccine, comprising a polypeptide according to claim 1 as an active ingredient or as an auxiliary ingredient for prevention of HIV 1 virus infection.

14. A vaccine, comprising a conjugate of the polypeptide according to claim 11 as an active ingredient or as an auxiliary ingredient for prevention of HIV 1 virus infection.

15. A complementary DNA sequence coding for the amino acid sequence of the polypeptide according to claim 1.

16. A host cell, characterized in that it contains at least one DNA sequence of claim 15.

17. The conjugate according to claim 7 for induction of HIV-1 virus-neutralizing antibodies in a subject in need thereof.

18. The conjugate according to claim 1 for prevention of diseases or disorders caused by HIV-1 virus.

19. The polypeptide according to claim 1, which has the length of up to 140 amino acids.

20. The polypeptide according to claim 1, which has the length of up to 120 amino acids.


SEQUENCE LISTING (60 sequences)

SEQ ID NO: 1 - protein, 111 aa, homo sapiens
KSELAVEILEKGQVRFWMQAEKLSGNAKVNYIFNEKEIFEGPKYKMHIDRNTGIIEMFMEKLQDEDEGTYTFQLQDGKATNHSTVVLVGDVFKKLQKEAEFQRQEWIRKQG

SEQ ID NO: 2 - protein, 111 aa, artificial
  mutated myomesin-1 domain 10 protein
  VARIANT (21)..(24): KAQQ, LSVF, ATPS, EIMW, DGSS, LLPL, WMWW, MNLY, MWRN, IMME, KHQL, HWQF, YAGN, or HGQW
  VARIANT (50)..(52): RNT, IMF, GHE, PSW, RAN, YFW, ITL, QAM, DMR, WLW, QGE, VQY, or VSL
  VARIANT (76)..(80): SHHLG, FMLMM, VILIL, IVTPL, DFIIW, MWSE(deletion), LYYAW, MMIEY, WMTQT, PQLWL, EPIFL, or QTATY
KSELAVEILEKGQVRFWMQAXXXXGNAKVNYIFNEKEIFEGPKYKMHIDXXXGIIEMFMEKLQDEDEGTYTFQLQXXXXXNHSTVVLVGDVFKKLQKEAEFQRQEWIRKQG

SEQ ID NO: 3 - protein, 111 aa, artificial
  mutated myomesin-1 domain 10 protein
KSELAVEILEKGQVRFWMQAKAQQGNAKVNYIFNEKEIFEGPKYKMHIDIMFGIIEMFMEKLQDEDEGTYTFQLQSHHLGNHSTVVLVGDVFKKLQKEAEFQRQEWIRKQG

SEQ ID NO: 4 - protein, 111 aa, artificial
  mutated myomesin-1 domain 10 protein
KSELAVEILEKGQVRFWMQALSVFGNAKVNYIFNEKEIFEGPKYKMHIDRNTGIIEMFMEKLQDEDEGTYTFQLQFMLMMNHSTVVLVGDVFKKLQKEAEFQRQEWIRKQG

SEQ ID NO: 5 - protein, 111 aa, artificial
  mutated myomesin-1 domain 10 protein
KSELAVEILEKGQVRFWMQAATPSGNAKVNYIFNEKEIFEGPKYKMHIDGHEGIIEMFMEKLQDEDEGTYTFQLQVILILNHSTVVLVGDVFKKLQKEAEFQRQEWIRKQG

SEQ ID NO: 6 - protein, 111 aa, artificial
  mutated myomesin-1 domain 10 protein
KSELAVEILEKGQVRFWMQAEIMWGNAKVNYIFNEKEIFEGPKYKMHIDPSWGIIEMFMEKLQDEDEGTYTFQLQIVTPLNHSTVVLVGDVFKKLQKEAEFQRQEWIRKQG

SEQ ID NO: 7 - protein, 111 aa, artificial
  mutated myomesin-1 domain 10 protein
KSELAVEILEKGQVRFWMQADGSSGNAKVNYIFNEKEIFEGPKYKMHIDRANGIIEMFMEKLQDEDEGTYTFQLQDFIIWNHSTVVLVGDVFKKLQKEAEFQRQEWIRKQG

SEQ ID NO: 8 - protein, 110 aa, artificial
  mutated myomesin-1 domain 10 protein
KSELAVEILEKGQVRFWMQALLPLGNAKVNYIFNEKEIFEGPKYKMHIDYFWGIIEMFMEKLQDEDEGTYTFQLQMWSENHSTVVLVGDVFKKLQKEAEFQRQEWIRKQG

SEQ ID NO: 9 - protein, 111 aa, artificial
  mutated myomesin-1 domain 10 protein
KSELAVEILEKGQVRFWMQAWMWWGNAKVNYIFNEKEIFEGPKYKMHIDITLGIIEMFMEKLQDEDEGTYTFQLQLYYAWNHSTVVLVGDVFKKLQKEAEFQRQEWIRKQG

SEQ ID NO: 10 - protein, 111 aa, artificial
  mutated myomesin-1 domain 10 protein
KSELAVEILEKGQVRFWMQAMNLYGNAKVNYIFNEKEIFEGPKYKMHIDQAMGIIEMFMEKLQDEDEGTYTFQLQMMIEYNHSTVVLVGDVFKKLQKEAEFQRQEWIRKQG

SEQ ID NO: 11 - protein, 111 aa, artificial
  mutated myomesin-1 domain 10 protein
KSELAVEILEKGQVRFWMQAMWRNGNAKVNYIFNEKEIFEGPKYKMHIDRNTGIIEMFMEKLQDEDEGTYTFQLQWMTQTNHSTVVLVGDVFKKLQKEAEFQRQEWIRKQG

SEQ ID NO: 12 - protein, 111 aa, artificial
  mutated myomesin-1 domain 10 protein
KSELAVEILEKGQVRFWMQAIMMEGNAKVNYIFNEKEIFEGPKYKMHIDDMRGIIEMFMEKLQDEDEGTYTFQLQIVTPLNHSTVVLVGDVFKKLQKEAEFQRQEWIRKQG

SEQ ID NO: 13 - protein, 111 aa, artificial
  mutated myomesin-1 domain 10 protein
KSELAVEILEKGQVRFWMQAKHQLGNAKVNYIFNEKEIFEGPKYKMHIDWLWGIIEMFMEKLQDEDEGTYTFQLQIVTPLNHSTVVLVGDVFKKLQKEAEFQRQEWIRKQG

SEQ ID NO: 14 - protein, 111 aa, artificial
  mutated myomesin-1 domain 10 protein
KSELAVEILEKGQVRFWMQAHWQFGNAKVNYIFNEKEIFEGPKYKMHIDQGEGIIEMFMEKLQDEDEGTYTFQLQPQLWLNHSTVVLVGDVFKKLQKEAEFQRQEWIRKQG

SEQ ID NO: 15 - protein, 111 aa, artificial
  mutated myomesin-1 domain 10 protein
KSELAVEILEKGQVRFWMQAYAGNGNAKVNYIFNEKEIFEGPKYKMHIDVQYGIIEMFMEKLQDEDEGTYTFQLQEPIFLNHSTVVLVGDVFKKLQKEAEFQRQEWIRKQG

SEQ ID NO: 16 - protein, 111 aa, artificial
  mutated myomesin-1 domain 10 protein
KSELAVEILEKGQVRFWMQAHGQWGNAKVNYIFNEKEIFEGPKYKMHIDVSLGIIEMFMEKLQDEDEGTYTFQLQQTATYNHSTVVLVGDVFKKLQKEAEFQRQEWIRKQG

SEQ ID NO: 17 - DNA, 29 bp, artificial
  Myom10-F
catatgaaatcagagttggcagttgaaat

SEQ ID NO: 18 - DNA, 33 bp, artificial
  Myom10-R
caagaatggatcaggaaacaaggttaaggatcc

SEQ ID NO: 19 - DNA, 70 bp, artificial
  Myom-LP-n1F
aatgccaaagtgaactatatcttcaacgagaaagaaatcttcgaaggtccgaaatacaaaatgcatattg

SEQ ID NO: 20 - DNA, 58 bp, artificial
  Myom-LP_2F
  misc_feature (28)..(36): randomized positions
ggtccgaaatacaaaatgcatattgatnnnnnnnnnggcatcatcgaaatgtttatgg

SEQ ID NO: 21 - DNA, 66 bp, artificial
  Myom-LP_n2R
cagctgaaaggtataggtgccttcatcttcatcctgcagtttttccataaacatttcgatgatgcc

SEQ ID NO: 22 - DNA, 58 bp, artificial
  Myom-LP_1F
  misc_feature (20)..(31): randomized positions
tcgtttttggatgcaggcannnnnnnnnnnnggtaatgccaaagtgaactatatcttc

SEQ ID NO: 23 - DNA, 60 bp, artificial
  Myom-LP_3R
  misc_feature (23)..(37): randomized positions
ccagaacaacggttgaatgattnnnnnnnnnnnnnnnctgcagctgaaaggtataggtgc

SEQ ID NO: 24 - DNA, 60 bp, artificial
  L-for
aaaagcgagctggccgtggaaattctggaaaaaggtcaggttcgtttttggatgcaggca

SEQ ID NO: 25 - DNA, 93 bp, artificial
  L-rev
accctgtttacgaatccattcttggcgctgaaattctgcttctttttgcagttttttgaacacgtcaccaaccagaacaacggttgaatgatt

SEQ ID NO: 26 - DNA, 87 bp, artificial
  JOIN-F
ctatagggagaccacaacggtttccctctagaaataattttgtttaactttaagaaggagatatacatatgaaaagcgagctggccg

SEQ ID NO: 27 - DNA, 40 bp, artificial
  JOIN-R
gaaccgaccgcggatccaccctgtttacgaatccattctt

SEQ ID NO: 28 - DNA, 41 bp, artificial
  T7B
atacgaaattaatacgactcactatagggagaccacaacgg

SEQ ID NO: 29 - DNA, 45 bp, artificial
  TolAk
ccgcacaccagtaaggtgtgcggtttcagttgccgctttctttct

SEQ ID NO: 30 - DNA, 19 bp, artificial
  P7 link
ggatccgcggtcggttcga

SEQ ID NO: 31 - DNA, 36 bp, artificial
  TolA rev
tttccgctcgagctacggtttgaagtccaatggcgc

SEQ ID NO: 32 - DNA, 61 bp, artificial
  His-Myo-F
cagtccatgggcagcagccatcatcatcatcatcacagcagcggcaaaagcgagctggccg

SEQ ID NO: 33 - protein, 4 aa, artificial
  part of SEQ ID NO. 2
KAQQ

SEQ ID NO: 34 - protein, 4 aa, artificial
  part of SEQ ID NO. 2
LSVF

SEQ ID NO: 35 - protein, 4 aa, artificial
  part of SEQ ID NO. 2
ATPS

SEQ ID NO: 36 - protein, 4 aa, artificial
  part of SEQ ID NO. 2
EIMW

SEQ ID NO: 37 - protein, 4 aa, artificial
  part of SEQ ID NO. 2
DGSS

SEQ ID NO: 38 - protein, 4 aa, artificial
  part of SEQ ID NO. 2
LLPL

SEQ ID NO: 39 - protein, 4 aa, artificial
  part of SEQ ID NO. 2
WMWW

SEQ ID NO: 40 - protein, 4 aa, artificial
  part of SEQ ID NO. 2
MNLY

SEQ ID NO: 41 - protein, 4 aa, artificial
  part of SEQ ID NO. 2
MWRN

SEQ ID NO: 42 - protein, 4 aa, artificial
  part of SEQ ID NO. 2
IMME

SEQ ID NO: 43 - protein, 4 aa, artificial
  part of SEQ ID NO. 2
KHQL

SEQ ID NO: 44 - protein, 4 aa, artificial
  part of SEQ ID NO. 2
HWQF

SEQ ID NO: 45 - protein, 4 aa, artificial
  part of SEQ ID NO. 2
YAGN

SEQ ID NO: 46 - protein, 4 aa, artificial
  part of SEQ ID NO. 2
HGQW

SEQ ID NO: 47 - protein, 5 aa, artificial
  part of SEQ ID NO. 2
SHHLG

SEQ ID NO: 48 - protein, 5 aa, artificial
  part of SEQ ID NO. 2
FMLMM

SEQ ID NO: 49 - protein, 5 aa, artificial
  part of SEQ ID NO. 2
VILIL

SEQ ID NO: 50 - protein, 5 aa, artificial
  part of SEQ ID NO. 2
IVTPL

SEQ ID NO: 51 - protein, 5 aa, artificial
  part of SEQ ID NO. 2
DFIIW

SEQ ID NO: 52 - protein, 4 aa, artificial
  part of SEQ ID NO. 2
MWSE

SEQ ID NO: 53 - protein, 5 aa, artificial
  part of SEQ ID NO. 2
LYYAW

SEQ ID NO: 54 - protein, 5 aa, artificial
  part of SEQ ID NO. 2
MMIEY

SEQ ID NO: 55 - protein, 5 aa, artificial
  part of SEQ ID NO. 2
WMTQT

SEQ ID NO: 56 - protein, 5 aa, artificial
  part of SEQ ID NO. 2
PQLWL

SEQ ID NO: 57 - protein, 5 aa, artificial
  part of SEQ ID NO. 2
EPIFL

SEQ ID NO: 58 - protein, 5 aa, artificial
  part of SEQ ID NO. 2
QTATY

SEQ ID NO: 59 - protein, 4 aa, homo sapiens
EKLS

SEQ ID NO: 60 - protein, 5 aa, homo sapiens
DGKAT